(19) United States
(12) Patent Application Publication
(10) Pub. No.: US 2026/0152812 A1
(43) Pub. Date: Jun. 4, 2026

(54) IDENTIFICATION OF A HUMAN CIRCOVIRUS

(71) Applicants: INSTITUT PASTEUR, PARIS (FR); UNIVERSITE PARIS CITE, PARIS (FR); ECOLE NATIONALE VETERINAIRE D'ALFORT, MAISON ALFORT (FR); ASSISTANCE PUBLIQUE - HOPITAUX DE PARIS, PARIS (FR)

(72) Inventors: Marc ELOIT, PARIS (FR); Philippe PEROT, PARIS (FR); Beatrice REGNAULT, PARIS (FR); Nicolas DA ROCHA, PARIS (FR); Anne JAMET, PARIS (FR); Jacques FOURGEAUD, PARIS (FR); Marianne LERUEZ-VILLE, PARIS (FR); Agnes FERRONI, PARIS (FR); Olivier LORTHOLARY, PARIS (FR); Claire ROUZAUD, PARIS (FR); Delphine CHRETIEN, PARIS (FR)

(21) Appl. No.: 18/865,909

(22) PCT Filed: May 17, 2023
(86) PCT No.: PCT/IB2023/000264

§ 371 Date: Nov. 14, 2024

Related U.S. Application Data
(60) Provisional application No. 63/343,205, filed on May 18, 2022.
(60) Provisional application No. 63/373,343, filed on Aug. 24, 2022.

(51) Int. Cl. C12Q 1/70; A61K 39/12; C07K 14/005; C07K 16/081; G01N 33/569

(52) U.S. Cl. CPC C12Q 1/701; A61K 39/12; C07K 14/005; C07K 16/081; G01N 33/56983; C12N 2750/10022; C12N 2750/10034; G01N 2333/01; G01N 2469/20

(57) ABSTRACT

The invention relates to the discovery of the first pathogenic human circovirus in samples from a human hepatitis patient, the capsid of which shows only 39% identity at the amino acid level with the closest known circovirus. This discovery allows for the generation of recombinant nucleic acids, primers, peptides, probes, antibodies, immunogenic compositions, and diagnostic assays specific for human circovirus.

### FIELD OF THE INVENTION

The invention relates to the detection or diagnosis of human circovirus infections comprising determining the presence of at least one nucleic acid or protein of said virus or antibodies thereto, in a biological sample. The invention also relates to the various diagnostic agents derived from the viral nucleic acids or proteins, in particular nucleic acid primers and probes, antigens and antibodies, and their use for the diagnosis of circovirus virus infection and associated disease, in particular hepatitis. The invention further relates to antigens derived from the viral proteins as vaccine for the prevention of circovirus infection and associated disease, in particular hepatitis.

### BACKGROUND OF THE INVENTION

Circoviruses are small viruses in the Circoviridae family with a circular single-stranded DNA genomes of ˜2 kb. Hui et al., Viruses 2021, 13, 944. Circoviruses are associated various asymptomatic to lethal diseases in birds, mammals, reptiles, and fish. Id. No pathogenic human circovirus has previously been described.

Human hepatitis is a potentially fatal disease that represents a health problem affecting millions of people worldwide associated with high mortality. While hepatitis A virus (HAV) causes acute self-limiting hepatitis, all hepatotropic viruses (hepatitis B, C, D, and E viruses (HBV, HCV, HDV, and HEV)), can produce chronic infections. According to the CDC, acute hepatitis non diagnosed as caused by a known virus can be caused by “exposure to some medications, drugs, alcohol, and toxins. It can also be caused by autoimmune disease. Other possible causes of non-viral hepatitis include contaminated water or food, dietary and herbal supplements, traditional or home remedies, wild-growing mushrooms and plants, and chemicals such as metals, solvents, paint thinners, or pesticides. Investigation of Acute Non-Viral Hepatitis of Unknown Etiology Potentially Associated with an Alkaline Water Product, C D C, 2021. In addition, still unknown viruses can be implicated. Therefore, the relevance of the diagnosis is essential for appropriate management of the disease. In this context, there is a need in the art for the identification of new pathogens responsible for hepatitis, which will improve the scope of possible diagnostic tests and increase diagnostic efficiency. The present invention fulfils this need in the art.

### BRIEF DESCRIPTION OF THE DRAWINGS

The invention is more fully understood by reference to the drawings.

FIG. 1 shows a phylogenetic analysis of the capsid gene of HCirV-1 with closest circovirus genus members.

FIG. 2 shows quality score (Q score) on sequences.

FIG. 3 shows the identification of Circoviridae sequences after patient's liver biopsy sequencing using Microseek and the closest viral sequences identified.

FIG. 4 shows clinical and laboratory data. Upper panel presents the monitoring of viral loads (HCirV-1, CMV, EBV, TTV), liver cytolysis markers (ALAT, ASAT, GGT) and blood cell count (Lymphocytes, White blood cells & Platelets) over time. The panel below summarizes the patient's treatment history.

FIG. 5 shows lobular hepatitis, apoptotic bodies, hepatocyte swelling and ballooning were present surrounded by a slight inflammatory infiltrate made of lymphocytes and histiocytes (G 20X, HES).

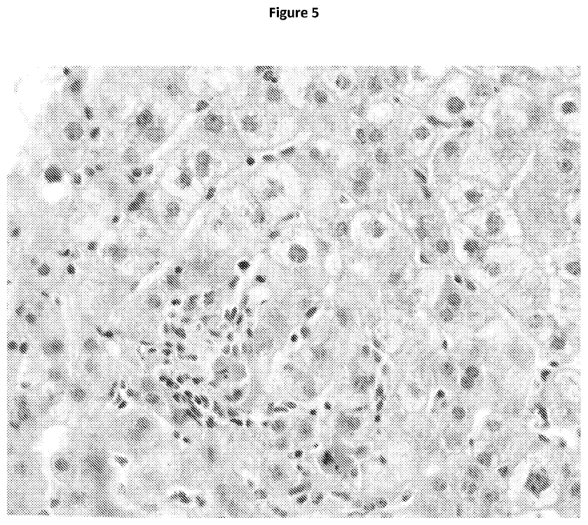

FIG. 6 shows phylogenetic analysis of HCirV-1. Phylogenetic analysis of capsid protein sequences of HCirV-1 and representative of circovirus strains. Sequences were aligned with MAFFT under the L-INS-I parameter, and maximum-likelihood phylogenetic reconstruction was performed with PhyML implemented through the NGPhylogeny portal. HCirV-1 capsid gene is depicted in red.

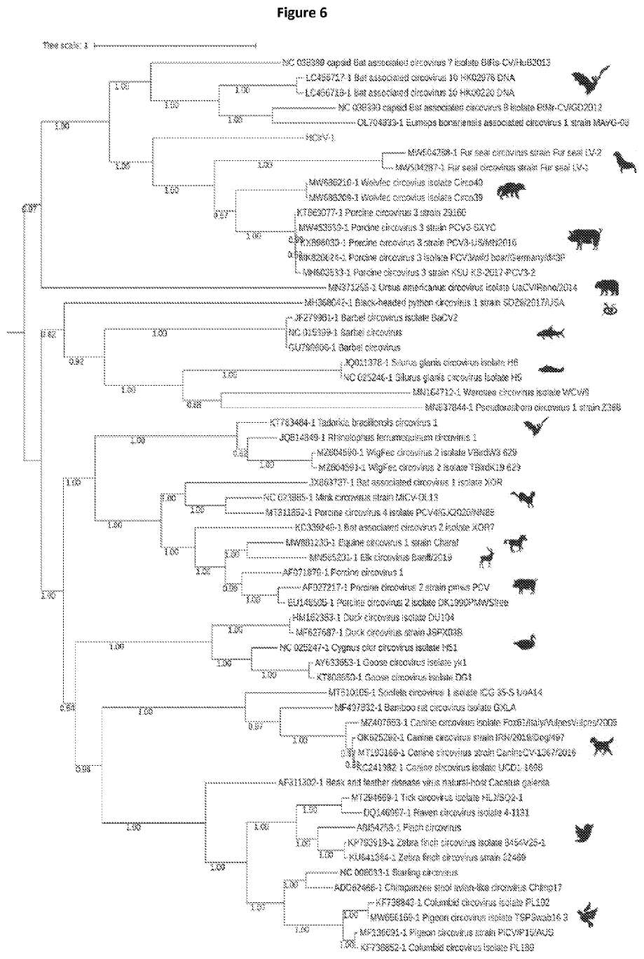

FIG. 7 shows in situ hybridization in liver section. Chromogenic in situ hybridization detection of HCirV-1 mRNA (labeled with red) in hepatocytes nuclei and cytoplasm. Nuclei were counterstained with Harris hematoxylin (Gx40).

### DETAILED DESCRIPTION OF THE INVENTION

A novel human circovirus was identified in samples from a human hepatitis patient. The capsid of the novel human circovirus shows only 39% identity at the amino acid level with the closest circovirus, Wolvfec circovirus, Genbank QSX73454. Phylogenetic analysis of the capsid gene shows that this virus corresponds to a new clade within the circovirus genus members.

The convergence of virological and clinical data show that the inventors have identified a new hepatotropic virus in France, belonging to a group of viruses represented till now only by animal pathogens. HCirV-1 could be responsible for less severe, non-labeled forms of hepatitis or other diseases in humans, or even other vertebrates.

The unique nature of HCirV-1 DNA and protein sequences allows for the generation of recombinant nucleic acids, expression vectors, primers, peptides, probes, antibodies, immunogenic compositions, and kits and diagnostic assays specific for human circovirus that do not amplify or detect other circovirus genus members.

### Recombinant Nucleic Acids

The invention encompasses recombinant nucleic acids comprising HCirV-1 nucleic acid sequences. In one embodiment, the recombinant nucleic acid comprises HCirV-1 capsid or replicase sequences.

In one embodiment, the recombinant nucleic acid of the invention comprises or consists of the following nucleic acid sequence, which corresponds to the full genome of HCirV-1, encoding the capsid protein and the replicase protein of HCirV-1:

(SEQ ID NO: 10)
CAGTATTACCCGGCACCTCGGAACCTGGAACCAGGAGCAAGATAGAGGTTTTCAGGTGTGG
CACTTGGCAGCCTATCAAGATGGATGGACCCGCCAAAAAGAGCAAGGTGCTGTATGGTGTT
CCTGTGGCAGTGCCGGGTCTGAATGTGAAATATGTTTTTGCTGATAATGTATTGTACTGGCAT
TGTGATGACTGTTATAAGTTTTGGCTGGATGAGAAGGAGATTCAATCTTATGTGTATTTGGAT
GCAGCGGGAGAGCCCAGTGGCGAGGTGGTGTTTTACGATAAACAACTGGACCCAAGAAGAG
TTGGATTGCTTGCTGCAGTGTGGCAAAGAGAAAGCGAAGTATCTTTGTATCGGCAGAGAGGT
TGGAAGCGGCGGTACCCGGCACTTGCAAGGATACGTGAATTTCAAGAACAAGCGAAGAATG
AGACAGGTAAAGGCCTTGCCTGGTTTTATGCGTGCTCACTTAGAAGCGGCCAAGGGGACCG
AGAAGCAGGCGAGCGACTACTGCAAGAAAGACGGCGATTACCTGGAGATTGGAGAGAGTGG
CTGTGCGGGGCGCCGACTGGATTTGGAGACAGCAGCGAAGATACTGACTGAGTCTGGCGG
GAACTTGAAGTGTGTTGCCGAGGCTTGTCCCGGGGTTTATATAAAGTATGGGGGGGGTTTGA
GGGATTACGCAAGTGTTATGAAATTAAAGCAACCTCGCAATTTTAAGAGTACTGTGATTGTTA
TATGTGGGGATCCTGGTTGTGGGAAAACTAGATATGTTATGGAGGATTGTAGGGAGAAAGG
GTTAAGCATGTATTGGAAGCCAAGGGGAATTTGGTGGGATGGCTATGATGGGGAGGATGTG
GTGGTGTATGATGATTTTTATGGGTGGGTTCCTTATGATGAGATGTTGAGGGTTATGGATAG
GTATCCTTTGAAGGTTCCTGTTAAGGGGGGTTATGTGGATTTTACATCTAAGATTATATATGT
GACAAGTAATGTGAAGCCTGAGGATTGGTATAGTAGTGAGAATATTAAGGGGAAGTTGGAGA
GTTTGTTTAGGCGGATCAATGTTTATCTGGAGGGTTTTGGCGGGGAGATCCGTGTGGGAAC
GCCTCTGTATCCTATTAACTATTAATAAAGACAAACAATGGTTGAAGTTCAACGGTTTGTTTAT
TTGGCGGGTACAATTGGCGGGAAGGAGGGAAAAATGGCGGGAGTTACTGACTGGGCCGCA
CAGCCGCCGCGGGGGTCCCCGACAGGGTCGGGGCGCGGGGGATCTTTTCCGGCCGCCCC
GTTAGGGCAGGAAAAGCGTTTACCTGGTCACTTGGGGCGGGTCACCCCCCTCGGAGTCAGT
TTATATAAGTTTGCTTAGAGAAGGTATTTCCATTTGATCCAGGCTGATTTGATGAGCCAGTAT
CTGACTGGTTGATTATATTCAACGTAATGAGAGTACTTGAGTCCTCCCCATTTGACAGTAGAT
TCTGCAGAGTTCCACCAGGTTCTGCGTGGGAGGAGAGCTGATGCTTGAGAAGAAGTAGTATT
GCCAGTAAGATGGATGAGAGGGCGAGGAGTAAAATATCTGCTATGCTTTCTGTTAGCCTTCC
AAAGTCTGGAAGTGGCAGTGTTGCAGAGTAGGTTTGATTGTACTGTGTTAGGTTCATTCCAG
GGTCCATCCAGGTCTATGATGGTGTTTCCTATAGGCCAGTAGTTCATTCGCTGTGTGGCATT
GAAGGGAAGGAAGGTTAGTTTGAGTTTTAGGATTTTATAAAAGTCCCAGCCTGGTGAGGAAA
CCCATTCACTGAGGGTGGTTGTGTAAGAGCCTTGCATCCATTTTTTGGCATTGGTTGAATCTT
GGTTTTGGAGGGTGAAGGGTTCAGTCTTGCAAAATTTTTGGACAACTGTTCCATTATTGGTGG
GACGTCTTATGTAAATGCGGCGTCTCCTGATTCGTCGAAATCTGCGGGCACGCCTCCTGGG
GCGGGGTCTTCTCCTCCTGAGGGTATATCTAGCCATGGTGCCGGT

This sequence has been now registered under the accession number ON677309.

In one embodiment, the invention encompasses variants thereof having more than 65%, 70%, 80%, 90%, 93%, 95%, 97%, 98%, 99% or 100% identity with SEQ ID NO: 10.

Particular variants are for example the hCirV-2 viruses whose sequences have been recently registered under accession numbers ON226770 and OP744467 (Li et al, Emerging Infectious Diseases, vol. 29, published on May 5, 2023). These variants share 68% and 79% identity with SEQ ID NO:10, respectively.

The recombinant nucleic acid of the invention can comprise all or at least 10, 20, 30, 40, 50, 60, 70, 80, 90, 100, 125, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 successive nucleotides identical to the nucleotide sequence SEQ ID NO: 10.

In a particular embodiment, the recombinant nucleic acid of the invention comprises or consists of the following nucleic acid sequence, which corresponds to the sequence of HCirV-1 encoding the capsid protein of HCirV-1:

(SEQ ID NO: 1)
ATGGCTAGATATACCCTCAGGAGGAGAAGACCCCGCCCCAGGAGGCGTGCCCGCAGATTT
CGACGAATCAGGAGACGCCGCATTTACATAAGACGTCCCACCAATAATGGAACAGTTGTCC
AAAAATTTTGCAAGACTGAACCCTTCACCCTCCAAAACCAAGATTCAACCAATGCCAAAAAA
TGGATGCAAGGCTCTTACACAACCACCCTCAGTGAATGGGTTTCCTCACCAGGCTGGGACT
TTTATAAAATCCTAAAACTCAAACTAACCTTCCTTCCCTTCAATGCCACACAGCGAATGAACT
ACTGGCCTATAGGAAACACCATCATAGACCTGGATGGACCCTGGAATGAACCTAACACAGT
ACAATCAAACCTACTCTGCAACACTGCCACTTCCAGACTTTGGAAGGCTAACAGAAAGCATA
GCAGATATTTTACTCCTCGCCCTCTCATCCATCTTACTGGCAATACTACTTCTTCTCAAGCAT
CAGCTCTCCTCCCACGCAGAACCTGGTGGAACTCTGCAGAATCTACTGTCAAATGGGGAGG
ACTCAAGTACTCTCATTACGTTGAATATAATCAACCAGTCAGATACTGGCTCATCAAATCAG
CCTGGATCAAATGGAAATACCTTCTCTAA

In one embodiment, the invention encompasses variants thereof having more than 65%, 70%, 80%, 90%, 93%, 95%, 97%, 98%, 99% or 100% identity with SEQ ID NO: 1.

In some embodiments, the recombinant nucleic acid of the invention comprises a fragment of SEQ ID NO: 1, comprising at least 10, 20, 30, 40, 50, 60, 70, 80, 90, 100, 125, 150, 200, 250, 300, 350, 400, 450, 500, 550, or 600 successive nucleotides of SEQ ID NO:1.

In another embodiment, the recombinant nucleic acid comprises or consists of the following nucleic acid sequence, encoding the replicase protein of HCirV-1:

(SEQ ID NO: 11)
ATGCAGCGGGAGAGCCCAGTGGCGAGGTGGTGTTTTACGATAAACAACTGGACCCAAGAAG
AGTTGGATTGCTTGCTGCAGTGTGGCAAAGAGAAAGCGAAGTATCTTTGTATCGGCAGAGAG
GTTGGAAGCGGCGGTACCCGGCACTTGCAAGGATACGTGAATTTCAAGAACAAGCGAAGAA
TGAGACAGGTAAAGGCCTTGCCTGGTTTTATGCGTGCTCACTTAGAAGCGGCCAAGGGGAC
CGAGAAGCAGGCGAGCGACTACTGCAAGAAAGACGGCGATTACCTGGAGATTGGAGAGAGT
GGCTGTGCGGGGCGCCGACTGGATTTGGAGACAGCAGCGAAGATACTGACTGAGTCTGGC
GGGAACTTGAAGTGTGTTGCCGAGGCTTGTCCCGGGGTTTATATAAAGTATGGGCGGGGTT
TGAGGGATTACGCAAGTGTTATGAAATTAAAGCAACCTCGCAATTTTAAGAGTACTGTGATTG
TTATATGTGGGGATCCTGGTTGTGGGAAAACTAGATATGTTATGGAGGATTGTAGGGAGAAA
GGGTTAAGCATGTATTGGAAGCCAAGGGGAATTTGGTGGGATGGCTATGATGGGGAGGATG
TGGTGGTGTATGATGATTTTTATGGGTGGGTTCCTTATGATGAGATGTTGAGGGTTATGGATA
GGTATCCTTTGAAGGTTCCTGTTAAGGGGGGTTATGTGGATTTTACATCTAAGATTATATATG
TGACAAGTAATGTGAAGCCTGAGGATTGGTATAGTAGTGAGAATATTAAGGGGAAGTTGGA
GAGTTTGTTTAGGCGGATCAATGTTTATCTGGAGGGTTTTGGCGGGGAGATCCGTGTGGGAA
CGCCTCTGTATCCTATTAACTATTAA

In one embodiment, the invention encompasses variants thereof having more than 65%, 70%, 75%, 80%, 85%, 90%, 93%, 95%, 97%, 98%, 99% or 100% identity with SEQ ID NO: 11.

The recombinant nucleic acid of the invention can comprise all or at least 10, 20, 30, 40, 50, 60, 70, 80, 90, 100, 125, 150, 200, 250, 300, 350, 400, 450, 500, 550, 600, 650, 700, 750, 800 or 850 successive nucleotides identical to the nucleotide sequence SEQ ID NO: 11.

In one embodiment, the recombinant nucleic acid of the invention comprises an origin or replication for replication of in bacteria or yeast.

In one embodiment, the recombinant nucleic acid of the invention comprises heterologous sequences allowing expression, such as a heterologous promoter or enhancer.

In one embodiment, the recombinant nucleic acid of the invention encodes the following amino acid sequence of the capsid protein of HCirV-1, or a fragment thereof:

(SEQ ID NO: 2)
MARYTLRRRRPRPRRRARRFRRIRRRRIYIRRPTNNGTVVQKFCKTEPF
TLQNQDSTNAKKWMQGSYTTTLSEWVSSPGWDFYKILKLKLTFLPFNAT
QRMNYWPIGNTIIDLDGPWNEPNTVQSNLLCNTATSRLWKANRKHSRYF
TPRPLIHLTGNTTSSQASALLPRRTWWNSAESTVKWGGLKYSHYVEYNQ
PVRYWLIKSAWIKWKYLL

In one embodiment, the recombinant nucleic acid of the invention encodes a protein having at least 70%, 75%, 80%, 85%, 90%, 93%, 95%, 97%, 98%, 99% or 100% identity with SEQ ID NO: 2.

In another embodiment, the recombinant nucleic acid of the invention encodes the following amino acid sequence of the replicase protein of HCirV-1, or a fragment thereof:

(SEQ ID NO: 12)
MQRESPVARWCFTINNWTQEELDCLLQCGKEKAKYLCIGREVGSGGTRH
LQGYVNFKNKRRMRQVKALPGFMRAHLEAAKGTEKQASDYCKKDGDYLE
IGESGCAGRRLDLETAAKILTESGGNLKCVAEACPGVYIKYGRGLRDYA
SVMKLKQPRNFKSTVIVICGDPGCGKTRYVMEDCREKGLSMYWKPRGIW
WDGYDGEDVVVYDDFYGWVPYDEMLRVMDRYPLKVPVKGGYVDFTSKII
YVTSNVKPEDWYSSENIKGKLESLFRRINVYLEGFGGEIRVGTPLYPIN
Y

In one embodiment, the recombinant nucleic acid of the invention encodes a protein with at least 70%, 75%, 80%, 85%, 90%, 93%, 95%, 97%, 98%, 99% or 100% identity with any of the sequences SEQ ID NO:2 or SEQ ID NO: 12.

### Expression Vectors

The invention encompasses a recombinant vector for expression of an HCirV-1 protein. The recombinant vector can be a vector for eukaryotic or prokaryotic expression, such as a plasmid, a phage for bacterium introduction, a YAC able to transform yeast, a viral vector and especially a retroviral vector, or any expression vector. An expression vector as defined herein is chosen to enable the production of an HCirV-1 protein or polyepitope, either in vitro or in vivo.

In one embodiment, the expression vector of the invention encodes a protein with at least 50%, 60%, 70%, 80%, 90%, 93%, 95%, 97%, 98%, 99% or 100% identity with SEQ ID NO: 2 or SEQ ID NO: 12.

In one embodiment, the expression vector of the invention encodes a protein with 50%, 60%, 70%, 80%, 90%, 93%, 95%, 97%, 98%, 99% or 100% identity with any of the sequences in SEQ ID NO:2 or SEQ ID NO: 12.

In one embodiment, the expression vector of the invention encodes a protein purification tag. In one embodiment, the expression vector encodes a protease cleavage site, such as TEV cleavage site, inserted between the HCirV-1 protein coding sequence and a protein purification tag, such as polyHis tag. In a preferred embodiment, the expression vector encodes a His tag. In one embodiment, a protease cleavage site is positioned to remove the His tag, for example, after purification.

The expression vector of the invention can comprise transcription regulation regions (including promoter, enhancer, ribosome binding site (RBS), polyA signal), a termination signal, a prokaryotic or eukaryotic origin of replication and/or a selection gene. The features of the promoter can be easily determined by the man skilled in the art in view of the expression needed, i.e., constitutive, transitory or inducible (e.g. IPTG), strong or weak, tissue-specific and/or developmental stage-specific promoter. The vector can also comprise sequence enabling conditional expression, such as sequences of the Cre/Lox system or analogue systems.

In various embodiments, the expression vector of the invention is a plasmid, a phage for bacterium introduction, a YAC able to transform yeast, a viral vector, or any expression vector. An expression vector as defined herein is chosen to enable the production of a protein or polyepitope, either in vitro or in vivo.

The nucleic acid molecules or the vectors according to the invention can be obtained by conventional methods, known per se, following standard protocols such as those described in Current Protocols in Molecular Biology (Frederick M. AUSUBEL, 2000, Wiley and son Inc., Library of Congress, USA). For example, they may be obtained by amplification of a nucleic sequence by PCR or RT-PCR or alternatively by total or partial chemical synthesis.

The vectors of the invention are constructed and introduced into host cells by conventional recombinant DNA and genetic engineering methods which are known per se. Numerous vectors into which a nucleic acid molecule of interest may be inserted in order to introduce it and to maintain it in a host cell are known per se; the choice of an appropriate vector depends on the use envisaged for this vector (for example replication of the sequence of interest, expression of this sequence, maintenance of the sequence in extrachromosomal form or alternatively integration into the chromosomal material of the host), and on the nature of the host cell.

The invention further encompasses cells comprising the vectors of the invention.

The invention also encompasses a method of preparing a protein comprising culturing cells comprising an expression vector of the invention and recovering the expressed protein.

The invention further encompasses the proteins produces by these methods from the nucleic acids of the invention.

### Primers and Probes

The invention encompasses primers and probes based on the HCirV-1 nucleic acid sequence of the invention, as described above. In one embodiment, the primers and probes of the invention are based on the HCirV-1 capsid or replicase sequence.

In one embodiment, the primers and probes of the invention comprise or consist of at least 10, 11, 12, 13, 14, 15, 16, 17, 18, 19, 20, 21, 22, 23, 24, 25, 26, 27, 28, 29, 30, 40, 50, 100, or 150 successive nucleotides identical to the nucleotide sequence of SEQ ID NO: 1.

Preferred primers for the amplification of HCirV-1 nucleic acid are:

HCirV1-Fw1:
(SEQ ID NO: 4)
ACCTGGATGGACCCTGGAAT,
HCirV1-Rv1:
(SEQ ID NO: 5)
AGAGTTCCACCAGGTTCTGC,
HCirV1-Fw2:
(SEQ ID NO: 6)
TTCGACGAATCAGGAGACGC,
HCirV1-Rv2:
(SEQ ID NO: 7)
GGCAGTGTTGCAGAGTAGGT,
HCirV1-Fw3:
(SEQ ID NO: 8)
CAGGAGACGCCGCATTTACA,
and
HCirV1-Rv3:
(SEQ ID NO: 9)
CCCATTCACTGAGGGTGGTT.

These three sets of PCR primers pairs (SEQ ID NO:4-5; SEQ ID NO:6-7 and SEQ ID NO:8-9) were designed in the capsid gene (i.e.: 6 individual primers corresponding to 3 forward primers and 3 reverse primers) in order to amplify short (<500 nt) fragments. Given that circoviruses are small circular DNA viruses, these primers can also be used in various combinations for inverse PCR experiments to amplify long (>500 nt) fragments. Alternatively, displacement amplification enzymes such as Phi29 polymerase can also be used for genome amplification, detection and genome finishing.

Other preferred TaqMan primers and probes for the amplification or labeling of HCirV-1 are the following:

HCirV-Fw:
(SEQ ID NO: 13)
5′-TTAGAAGCGGCCAAGGGGA-3′
HCirV-Rv:
(SEQ ID NO: 14)
5′-CGCTGCTGTCTCCAAATCCA-3′
HCirV-Pb:
([FAM]-SEQ ID NO: 15-[BHQ])
5′-[FAM]GGAGATTGGAGAGAGTGGCTG[BHQ1]-3′

The sequences of these primers and probes have been designed so as to detect the Replicase protein of human circoviruses specifically, without detecting other non-human circoviruses. The resulting amplicon has a size of about 120 base pairs.

Preferably, the primer or probe of the invention is labeled with a fluorescent, radioactive, or enzymatic label.

The primer and/or probe of the invention can comprise or consist of all or at least 10, 11, 12, 13, 14, 15, 16, 17, 18, 19, 20, 21, 22, 23, 24, 25, 26, 27, 28, 29, 30, 40, 50, 100, or 150 successive nucleotides identical to any of the nucleotide sequences SEQ ID NO:1, SEQ ID NO: 10 or SEQ ID NO: 11.

The primer and/or probe of the invention can comprise or consist of all or at least 10, 11, 12, 13, 14, 15, 16, 17, 18, 19, 20, or 21 of SEQ ID NO: 4-9 or of SEQ ID NO: 13-15.

The invention encompasses the primers and probes of the invention for use in diagnostic assays and their use in these assays.

### Nucleic Acid Detection and Amplification Kits

The invention encompasses nucleic acid detection and amplification kits containing a probe and/or primer of the invention, such as those disclosed above.

Preferably, the primer or probe of the invention is labeled with a fluorescent, radioactive, or enzymatic label.

Preferably, the kit of the invention comprises amplification and/or hybridization reagents. For example, the kit of the invention can contain dNTPs (dATP, dCTP, dGTP and dTTP), a buffer, and a reverse transcriptase and/or heat-stable polymerase (such as Taq Polymerase).

### Nucleic Acid Diagnostic Assays

The invention encompasses nucleic acid diagnostic assays utilizing a probe and/or primer of the invention.

The invention encompasses methods for specific detection of HCirV-1.

In one embodiment, the method of the invention comprises providing a sample, contacting the sample with a probe of the invention and detecting the presence or absence of HCirV-1 nucleic acid in the sample by routine techniques in the art.

In one embodiment, the sample can be subject to an amplification reaction to increase the amount of HCirV-1 nucleic acid in the sample for detection.

In one embodiment, the method of the invention comprises providing a sample, optionally subjecting the sample to a reverse transcription reaction to generate a cDNA copy of HCirV-1 RNA in the sample using a “reverse primer” specific for HCirV-1 RNA, amplifying HCirV-1 DNA with a “reverse primer” and a “forward primer,” and detecting any amplified product. In one embodiment, the amplified product is detected with a probe. The method can be used for the determination of whether or not HCirV-1 is present in the sample.

Preferred primer sets for the amplification of HCirV-1 nucleic acid are:
HCirV1-Fw1 (SEQ ID NO: 4) with HCirV1-Rv1 (SEQ ID NO: 5),
HCirV1-Fw2 (SEQ ID NO: 6) with HCirV1-Rv2 (SEQ ID NO: 7), and
HCirV1-Fw3 (SEQ ID NO: 8) with HCirV1-Rv3 (SEQ ID NO: 9).

Other preferred primer sets are:

HCirV-Fw:
(SEQ ID NO: 13)
5′-TTAGAAGCGGCCAAGGGGA-3′
HCirV-Rv:
(SEQ ID NO: 14)
5′-CGCTGCTGTCTCCAAATCCA-3′

In preferred embodiments of the assay of the invention, the sample is a biological sample, for example, stool, saliva, blood, plasma, serum, urine, cerebrospinal fluid, or tissue, preferably liver, sample. In some embodiments, the sample is a human or animal clinical sample (i.e., a sample from living or dead individual (human or animal) suspected of having a circovirus virus. In another embodiment, the sample can be environmental (sewage).

The sample can be subjected to well-known isolation and purification protocols or used directly. For example, the sample can be subjected to a treatment to release/extract the nucleic acids of the sample and/or to remove proteins and other non-nucleic acid components of the sample using conventional techniques.

Amplification of HCirV-1 genomic DNA can be performed with two primers, a “forward” and a “reverse” primer, both of which are specific for HCirV-1.

Reverse transcription of the RNA of a HCirV-1 can be performed with a “reverse primer’ specific for HCirV-1. A “reverse primer’ is one that, based on its 5′-3′ orientation, can bind to a single-stranded RNA and serve to initiate generation of a complementary DNA (cDNA) copy of the RNA. The reverse transcription can be accomplished using well known and routine methods. The reaction mix for reverse transcription contains the reagents for the reaction, for example, a reverse primer, dNTPs (dATP, dCTP, dGTP and dTTP), a buffer, and a reverse transcriptase. Exemplary reaction conditions are set forth in the examples.

Amplification of the cDNA copy of a HCirV-1 generated by reverse transcription can be performed with a “forward primer’ specific for HCirV-1. A “forward primer’ is one that, based on its 5′-3′ orientation, can bind to a single-stranded antisense cDNA copy of an RNA generated by reverse transcription and serve to initiate generation of a double-stranded DNA copy of the RNA. The amplification can be accomplished using well known and routine methods. The reagent mix for amplification contains the reagents for the reaction, for example a forward primer, a reverse primer, dNTPs, a buffer, and a DNA polymerase.

In one embodiment, the method of the invention is performed using a single RT-PCR reagent mix containing the reagents for the reverse transcription and amplification reactions. Preferably, the reverse primer used for the reverse transcription reaction is also used for the amplification reaction.

Preferably, the reverse transcription and amplification reactions are performed in a plastic or glass container, most preferably in the same container.

Amplification methods known in the art include RCA, MDA, NASBA, TMA, SDA, LCR, b-DNA, PCR (all forms including RT-PCR), RAM, LAMP, ICAN, SPIA, QB-replicase, or Invader. A preferred amplification method is the polymerase chain reaction (PCR) amplification. See, e.g., PCR Technology: Principles and Applications for DNA Amplification (Ed. H. A. Erlich, Freeman Press, NY, N.Y., 1992); PCR Protocols: A Guide to Methods and Applications (Eds. Iinis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675. More preferred PCR methods is real-time PCR, PCR-HRM (High-Resolution DNA Melting) (see Andriantsoanirina et al. Journal of Microbiological Methods, 78:165 (2009)) and PCR coupled to ligase detection reaction based on fluorescent microsphere (Luminex® microspheres).

Amplification techniques include in particular isothermal methods and PCR-based techniques. Isothermal techniques include such methods as nucleic acid sequence-based amplification (NASBA), loop-mediated isothermal amplification (LAMP), helicase-dependent amplification (HDA), rolling circle amplification (RCA), and strand displacement amplification (SDA), exponential amplification reaction (EXPAR), isothermal and chimeric primer-initiated amplification of nucleic acids (ICANs), signal-mediated amplification of RNA technology (SMART) and others (see e.g. Asiello and Baeumner, Lab Chip; 11 (8): 1420-1430, 2011).

Preferably, the PCR technique quantitatively measures starting amounts of DNA, cDNA, or RNA. Examples of PCR-based techniques according to the invention include techniques such as, but not limited to, quantitative PCR (Q-PCR), reverse-transcriptase polymerase chain reaction (RT-PCR), quantitative reverse-transcriptase PCR (QRT-PCR), or digital PCR. These techniques are well known and easily available technologies for those skilled in the art. Preferably, the Q-PCR is performed with the primers set forth in the examples, preferably as set forth in the examples herein.

Preferably, the method of the invention is a one-step real-time RT-PCR assay. Preferably, a probe is used to detect the amplified product. The probe can be labeled with a fluorescent, radioactive, or enzymatic label. The amplified product can be detected with a specific detection chemistry such as fluorescence resonance energy transfer (FRET) probes, TAQMAN probes, molecular beacons, scorpion probes, fluorescently labeled (or other labeled) primers, lightup probes or a dye-based chemistry, DNA, PNA, LNA, or RNA including modified bases that bind to the amplified product to detect the sequence of interest.

Detection of the amplified products can be real-time (during the amplification process) or endpoint (after the amplification process). The invention allows for detection of the amplification products in the same vessel as amplification occurs.

Preferably, a DNA internal control is used to monitor the amplification reaction.

Preferably, an RNA internal control is used to monitor the reverse transcription and amplification reactions.

The primers of the invention are useful for both reverse transcription of HCirV-1 RNA and amplification of HCirV-1DNA. The primer sequences are preferably selective to HCirV-1.

The invention encompasses a set of primers, i.e., at least two primers of different orientations. Preferably, the primers are in a set of one forward primer and one reverse primer. All of the primers referred to herein can be specifically included in this set of primers.

The “reverse primer’ is an anti-sense primer, which can be the primer for reverse transcription, and preferably does not bind to non-human circoviruses, but is specific for HCirV-1.

In one embodiment, RNA sequences of HCirV-1 can be detected, preferably in cells, for example by ISH. In one embodiment, cocktails of bDNA probes can be designed to target the capsid and/or Rep genes of HCirV-1, for example as set forth in the examples. In one embodiment, the ViewRNA ISH Tissue Assay Kit 2-plex (Thermo Fisher Scientific) can be used.

A preferred DNA probe is for example:

HCirV-Pb:
([FAM]-SEQ ID NO: 15-[BHQ])
5′-[FAM]GGAGATTGGAGAGAGTGGCTG[BHQ1]-3′

### Proteins and Peptides

The invention encompasses proteins and peptides based on the HCirV-1 amino acid sequence. In one embodiment, the protein or peptide of the invention is an HCirV-1 capsid or replicase protein or peptide. In one embodiment, the protein or peptide of the invention comprises or consists of at least 6, 7, 8, 9, 10, 11, 12, 13, 14, 15, 16, 17, 18, 19, 20, 30, 40, 50, 60, 70, 80, 90, 10, 125, 150, 175, 200, 225 or 250 amino acids of SEQ ID NO: 2 (capsid protein) or SEQ ID NO: 12 (replicase protein).

In some embodiments, the protein or peptide of the invention comprises or consists of all or at least 6, 7, 8, 9, 10, 11, 12, 13, 14, 15, 16, 17, 18, 19, 20, 30, 40, 50, 60, 70, 80, 90, 10, 125, 150, 175, 200, 225 or 250 amino acids of any of the sequences SEQ ID NO: 2 or SEQ ID NO: 12. These peptides are herein called the “fragments” of the invention.

In some embodiments, the protein or peptide of the invention are variants thereof having at least 70%, 75%, 80%, 85%, 90%, 93%, 95%, 97%, 98%, 99% or 100% identity with SEQ ID NO: 2 or SEQ ID NO: 12.

In a preferred embodiment, the following capsid protein fragments can be used for detecting the presence of the virus by serologic means:

(SEQ ID NO: 16)
NNGTVVQKFCKTEPFTLQNQDSTNAKKWMQGSYTTTLSEWVSSPGWDFY
KILKLKLTFLPFNATQRMNYWPIGNTIIDLDGPWNEPNTVQSNLLCNTA
TSRLWKANRKHSRYFTPRPLIHLTGNTTSSQASALLPRRTWWNSAESTV
KWGGLKYSHYVEYNQPVRYWLIKSAWIKWKYLL
(SEQ ID NO: 17)
NNASIVQKFCKWEPISFQNTTNQNKWYIGSYTTNLKEWVTQPNWDLYKI
LKMKISFLPFTPTQKMPYWPIGNTIIDLDGPWPQPYTANSNLFCNTATS
RSWYGNKKHSRYFTPRAVVNLTTTATTGEGTALLPRSTWWNCNNDSIIW
GGLRFAHYIENGSNMAIRYHLQKSVWIKWKYLL

This latter sequence corresponds to a fragment of the capsid protein of the human Circovirus obtained from a second patient, YN09/J030, by the team of Li et al, Emerging Infectious Diseases, vol. 29, published on May 5, 2023.

In some embodiments, the protein or peptide of the invention comprises or consists of all or at least 6, 7, 8, 9, 10, 11, 12, 13, 14, 15, 16, 17, 18, 19, 20, 30, 40, 50, 60, 70, 80, 90, 10, 125, 150, or 175 amino acids of any of the sequences SEQ ID NO: 16 or SEQ ID NO: 17. These peptides are herein also called the “fragments” of the invention.

Preferably, the protein or peptide of the invention is labeled. In one embodiment, the protein or peptide or fragment of the invention is labeled with a visualizing molecule, such as a radioactive atom, a dye, a fluorescent molecule, a fluorophore, an enzyme, colloidal gold, a magnetic particle, or a latex bead.

The invention encompasses the protein and peptide of the invention for use in diagnostic assays and their use in these assays.

### Antibodies

The invention encompasses antibodies generated against an HCirV-1 protein sequence as described above. In one embodiment, the antibody binds to the protein of SEQ ID NO: 2 or of SEQ ID NO: 12 or of SEQ ID NO: 16/17. Preferably, the antibody does not bind to the capsid protein of any other circovirus like Wolvfec circovirus (Genbank QSX73454, SEQ ID NO: 3) nor to the capsid protein of Porcine circovirus 3.

The invention encompasses the antibodies of the invention for use in diagnostic assays and their use in these assays.

In one embodiment, the polyclonal or monoclonal antibodies of the invention are generated in rabbits or mice.

In one embodiment, the VHH antibodies of the invention are generated, for example in alpaca.

The invention encompasses a polyclonal or monoclonal antibody or fragment thereof directed against an HCirV-1 protein or peptide or fragment as described above (SEQ ID NO:2, 12, 16, 17 or any variant or fragment thereof as described above).

In one embodiment, the antibodies of the invention can be obtained by immunization of an animal with an HCirV-1 protein.

The antibodies of the invention can serve as reagents to bind native HCirV-1 protein of patients in immunoassays.

The antibodies of the invention can serve as positive control reagents to bind isolated and purified HCirV-1 proteins in immunoassays of patients.

The invention of the invention encompasses the polyclonal antibodies, monoclonal antibodies, chimeric antibodies, and fragments thereof (e.g., Fab, Fv, scFv) directed against HCirV-1 proteins.

For the purposes of the present invention, the expression chimeric antibody is understood to mean, in relation to an antibody of a particular animal species or of a particular class of antibody, an antibody comprising all or part of a heavy chain and/or of a light chain of an antibody of another animal species or of another class of antibody.

In some embodiments, purified proteins are used to produce antibodies by conventional techniques. In some embodiments, recombinant or synthetic proteins or peptides are used to produce antibodies by conventional techniques.

Antibodies can be synthetic, semi-synthetic, monoclonal, or polyclonal and can be made by techniques well known in the art. Such antibodies specifically bind to proteins and polypeptides via the antigen-binding sites of the antibody (as opposed to non-specific binding). Purified or synthetic proteins and peptides can be employed as immunogens in producing antibodies immunoreactive therewith. The proteins and peptides contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes can be identified by any of the methods known in the art. Such epitopes or variants thereof can be produced using techniques well known in the art such as solid-phase synthesis, chemical or enzymatic cleavage of a polypeptide, or using recombinant DNA technology.

Antibodies are defined to be specifically binding if they bind proteins or polypeptides with a Ka of greater than or equal to about 10⁷ M⁻¹. Affinities of binding partners or antibodies can be readily determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, alpaca, camels, rabbits, mice, or rats, using procedures that are well known in the art. In general, a purified protein or polypeptide that is appropriately conjugated is administered to the host animal typically through parenteral injection. The immunogenicity can be enhanced through the use of an adjuvant, for example, Freund's complete or incomplete adjuvant. Following booster immunizations, small samples of serum are collected and tested for reactivity to proteins or polypeptides. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, Mckeam, and Bechtol (eds.), 1980.

For example, the host animals, such as mice, can be injected intraperitoneally at least once and preferably at least twice at about 3 week intervals with isolated and purified proteins or conjugated polypeptides, for example a peptide comprising or consisting of the specific amino acids set forth above. Mouse sera are then assayed by conventional dot blot technique or antibody capture (ABC) to determine which animal is best to fuse. Approximately two to three weeks later, the mice are given an intravenous boost of the protein or polypeptide. Mice are later sacrificed, and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as a labeled protein or polypeptide, is added to each well followed by incubation. Positive wells can be subsequently detected. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

Antigen-binding fragments of such antibodies, which can be produced by conventional techniques, are also encompassed by the present invention. Examples of such fragments include, but are not limited to, Fab and F(ab′) 2 fragments. Antibody fragments and derivatives produced by genetic engineering techniques are also provided.

The monoclonal antibodies of the present invention include chimeric antibodies, e.g., humanized versions of murine monoclonal antibodies. Such humanized antibodies can be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May 1993). Procedures to generate antibodies transgenically can be found in GB 2,272,440, U.S. Pat. Nos. 5,569,825 and 5,545,806.

Antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Publication No. WO 87/02671; Akira, et al. European Patent Application 0184187; Taniguchi, M., European Patent Application 0171496; Morrison et al. European Patent Application 0173494; Neuberger et al. PCT International Publication No. WO 86/01533; Cabilly et al. U.S. Pat. No. 4,816,567; Cabilly et al. European Patent Application 0125023; Better et al., Science 240:1041 1043, 1988; Liu et al., PNAS 84:3439 3443, 1987; Liu et al., J. Immunol. 139:3521 3526, 1987; Sun et al. PNAS 84:214 218, 1987; Nishimura et al., Canc. Res. 47:999 1005, 1987; Wood et al., Nature 314:446 449, 1985; and Shaw et al., J. Natl. Cancer Inst. 80:1553 1559, 1988); Morrison, S. L., Science 229:1202 1207, 1985; Oi et al., BioTechniques 4:214, 1986; Winter U.S. Pat. No. 5,225,539; Jones et al., Nature 321:552 525, 1986; Verhoeyan et al., Science 239:1534, 1988; and Beidler et al., J. Immunol. 141:4053 4060, 1988.

In connection with synthetic and semi-synthetic antibodies, such terms are intended to cover but are not limited to antibody fragments, isotype switched antibodies, humanized antibodies (e.g., mouse-human, human-mouse), hybrids, antibodies having plural specificities, and fully synthetic antibody-like molecules.

In one embodiment, the invention encompasses single-domain antibodies (sdAb), also known as NANOBODIES. A sdAb is a fragment consisting of a single monomeric variable antibody domain that can bind selectively to a specific antigen.

In one embodiment, the sdAbs of the invention are from heavy-chain antibodies found in camelids (VHH fragments), or cartilaginous fishes (VNAR fragments), or are obtained by splitting dimeric variable domains into monomers.

### Serological and Virological Diagnosis

The HCirV-1 proteins and the peptides derived from these proteins and antibodies generated against them, as described above, can be used for the detection and diagnosis of a human circovirus infection (serological diagnosis (detection of specific antibodies) or virological diagnosis (detection of viral protein), in particular by an immunoassay, such as an immunoenzymatic method (e.g., ELISA).

The invention encompasses methods for identifying a patient infected with a circovirus, comprising providing a serum sample from the patient, contacting the serum with an HCirV-1 protein, and visualizing the antigen-antibody complexes. Preferably, the antigen-antibody complexes are visualized by EIA, ELISA, RIA, or by immunofluorescence.

The invention encompasses a composition comprising an HCirV-1 protein or the use of an HCirV-1 protein for detection of antibodies against a circovirus and diagnosis of a circovirus infection in a biological sample.

The antibodies and antibody fragments according to the invention directed to HCirV-1 proteins and the derived peptides, are useful for the direct detection and diagnosis of a circovirus infection and for the detection of a HCirV-1 in a biological sample. The detection of the capsid or replicase protein of a HCirV-1 is carried out by an appropriate technique, in particular EIA, ELISA, RIA, immunofluorescence, in a biological sample collected from an individual likely to be infected.

In one embodiment, the invention comprises a method for the detection of a human circovirus in a biological sample, which method is characterized in that it comprises contacting the biological sample from a patient infected with a circovirus with an anti-HCirV-1 antibody, and visualizing the antigen-antibody complexes formed. Preferably, the antigen-antibody complexes are visualized by EIA, ELISA, RIA, or by immunofluorescence.

In one embodiment, the HCirV-1 protein of the invention is attached to an appropriate support, in particular a microplate or a bead.

In one embodiment, the method of the invention comprises bringing a biological sample from a subject, preferably a human, infected with a circovirus into contact with an HCirV-1 protein, which is attached to an appropriate support, in particular a microplate or bead, to allow binding to occur; washing the support to remove unbound antibodies; adding a detection reagent that binds to the immunoglobulins bound to HCirV-1 protein; and detecting the HCirV-1 protein-antibody complexes formed.

In one embodiment, the method of the invention for the detection of antibodies against a circovirus in a biological sample comprises providing a HCirV-1 protein; providing a biological sample from a patient infected with a circovirus; contacting said HCirV-1 protein with said biological sample; and visualizing the antigen-antibody complexes formed. Preferably, the method comprises an ELISA.

Preferably, the protein-antibody complexes are detected with an antibody or an antibody fragment that binds to human immunoglobulins.

Preferably, the detection reagent comprises a label is selected from a chemiluminescent label, an enzyme label, a fluorescence label, and a radioactive (e.g., iodine) label. Most preferably, the detection reagent is a labeled antibody or antibody fragment that binds to human immunoglobulins.

Preferred labels include a fluorescent label, such as FITC, a chromophore label, an affinity-ligand label, an enzyme label, such as alkaline phosphatase, horseradish peroxidase, luciferase or galactosidase, an enzyme cofactor label, a hapten conjugate label, such as digoxigenin or dinitrophenyl, a Raman signal generating label, a magnetic label, a spin label, an epitope label, such as the FLAG or HA epitope, a luminescent label, a heavy atom label, a nanoparticle label, an electrochemical label, a light scattering label, a spherical shell label, semiconductor nanocrystal label, wherein the label can allow visualization with or without a secondary detection molecule.

Preferred labels include suitable enzymes such as horseradish peroxidase, alkaline phosphatase, beta-galactosidase, luciferase or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, as well as others known in the art; a luminescent material such as luminol; light scattering or plasmon resonant materials such as gold or silver particles or quantum dots; or radioactive material include ¹⁴C, ¹²³I, ¹²⁴I, ¹²⁵I, ³²P, ³³P, ³⁵S, or ³H.

In one embodiment, the antibody or an antibody fragment of the invention that binds to human immunoglobulins binds specifically to IgG, IgA, and IgM. In one embodiment, the antibody or an antibody fragment of the invention that binds to human immunoglobulins binds specifically to IgG, IgA, or IgM.

The term “antibodies” is meant to include polyclonal antibodies, monoclonal antibodies, fragments thereof, such as F(ab′)2 and Fab fragments, single-chain variable fragments (scFvs), single-domain antibody fragments (VHHs or Nanobodies), bivalent antibody fragments (diabodies), as well as any recombinantly and synthetically produced binding partners.

In a preferred embodiment, the antibody of the invention is a VHH, preferably an alpaca serum.

In one embodiment, the method of the invention comprises comparing the results obtained with a patient serum to positive and negative controls.

Positive controls can include:
Serum from animals (e.g., rabbit, alpaca, etc.) immunized with HCirV-1 protein as described above,
An HCirV-1 protein as described above, preferably comprising SEQ ID NO: 2.

The method of the invention can comprise the use of HCirV-1 proteins to detect novel circoviruses that do not cross-react with circoviruses of other species.

In one embodiment, the method of the invention comprises an immunocapture method.

In one embodiment, the method of the invention comprises attaching a first monoclonal or polyclonal antibody or a fragment thereof, directed against the HCirV-1 protein (capture antibody), incubating the antibody with a biological sample containing a HCirV-1 protein, and detecting the antigen-antibody complexes formed, preferably with a monoclonal antibody (visualizing antibody).

In one embodiment, the biological sample used in the method of the invention is mixed with the visualizing monoclonal antibody prior to its being brought into contact with the capture antibody.

In the immunocapture tests according to the invention, it is possible to use, for visualizing the HCirV-1 protein, a monoclonal antibody conjugated with a visualizing molecule or particle.

A visualizing molecule may be a radioactive atom, a dye, a fluorescent molecule, a fluorophore, an enzyme; a visualizing particle may be for example: colloidal gold, a magnetic particle or a latex bead.

The subject of the present invention is also an immune complex formed of a polyclonal or monoclonal antibody or antibody fragment as defined above, and of a HCirV-1 protein.

### Antigen and Antibody Detection Kits

The invention encompasses a circovirus detection kit, characterized in that it comprises a HCirV-1 protein, as described above, and/or antibodies generated against them.

In one embodiment, the HCirV-1 protein of the invention comprises the amino acid sequence of SEQ ID NO: 2 or SEQ ID NO: 12 or SEQ ID NO: 16/17 or fragments or variants thereof as described above.

In one embodiment, the invention comprises a kit for the detection of a circovirus infection, which kit contains a HCirV-1 protein and reagents as described above, for detection of antigen-antibody complexes.

Preferably, the kit of the invention contains a serum of an animal immunized with a HCirV-1 protein.

Most preferably, the serum of the invention is a rabbit or alpaca serum from an animal immunized with a HCirV-1 protein.

In one embodiment, the kit of the invention comprises an HCirV-1 protein that comprises or consists of the amino acid sequence of SEQ ID NO: 2, or of SEQ ID NO: 12 or of SEQ ID NO: 16 or of SEQ ID NO: 17, or fragments or variants thereof as described above.

In one embodiment, the kit of the invention comprises both HCirV-1 protein and an HCirV-1 immune serum.

In one embodiment, the kit of the invention is a Simple/Rapid test designed for use where a preliminary screening test result is required. The tests can be a test based on agglutination, immuno-dot, immuno-chromatographic and/or immuno-filtration techniques. Preferably, the test is quick and easy to perform, preferably from about 10 minutes to about 2 hours, and requires little or no additional equipment.

Preferably, the kit of the invention can be stored at room temperature for extended period of time.

### Immunogenic Compositions

The present invention also relates to an immunogenic composition or vaccine composition comprising a peptide or protein according to the invention, e.g., of SEQ ID NO:2, SEQ ID NO: 12, SEQ ID NO: 16 or SEQ ID NO: 17, or any variant/fragment thereof.

In a particular embodiment, the immunogenic composition of the invention further comprises an adjuvant and/or a pharmaceutically acceptable vehicle.

As defined herein, a pharmaceutically acceptable vehicle encompasses any substance that enables the formulation of the polyepitope, the polynucleotide, the vector according to the invention within a composition. A vehicle is any substance or combination of substances physiologically acceptable i.e., appropriate for its use in a composition in contact with a host, especially a human, and thus non-toxic. Examples of such vehicles are phosphate buffered saline solutions, distilled water, emulsions such as oil/water emulsions, various types of wetting agents sterile solutions and the like.

As defined herein, an adjuvant includes, for example, liposomes, oily phases, such as Freund type adjuvants, generally used in the form of an emulsion with an aqueous phase or can comprise water-insoluble inorganic salts, such as aluminium hydroxide, zinc sulphate, colloidal iron hydroxide, calcium phosphate or calcium chloride.

In another particular embodiment, the immunogenic composition of the invention is formulated for an administration through parenteral route such as subcutaneous (s.c.), intradermal (i.d.), intramuscular (i.m.), intraperitoneal (i.p.) or intravenous (i.v.) injection.

In another particular embodiment, the immunogenic composition of the invention is administered in one or multiple administration dose(s), in particular in a prime-boost administration regime.

The quantity to be administered (dosage) depends on the subject to be treated, including the condition of the patient, the state of the individual's immune system, the route of administration and the size of the host. Suitable dosages range from 10³ TCID50 to 10⁷ TCID50 for a viral vector or 100 micrograms of plasmid DNA, and can be modified by one skilled in the art, depending on circumstances.

In a preferred embodiment, the immunogenic or vaccine composition of the invention is for use in the prevention of a circovirus infection and associated disease, in particular hepatitis, in a human subject.

### EXAMPLES

We report the case of a 61-year-old solid-organ transplanted woman presenting with cytolytic hepatitis of unknown etiology. The patient underwent a heart-lung transplant 17 years ago for Eisenmenger syndrome related to ventricular septal defect. The maintenance immunosuppressive regimen included tacrolimus, mycophenolate mofetil and prednisone. She had pulse steroids 16 months before the hepatitis occurrence for acute lung allograft dysfunction. The patient had a history of localized left upper lobe pulmonary adenocarcinoma treated by surgery two and a half years before presentation. She also had a history of chronic renal failure due to calcineurin inhibitor (Cockcroft-gault to 23 mL/min), Aspergillus sp. and Scedosporium sp. bronchopulmonary infections, Parvovirus B19 infection and a COVID-19 infection.

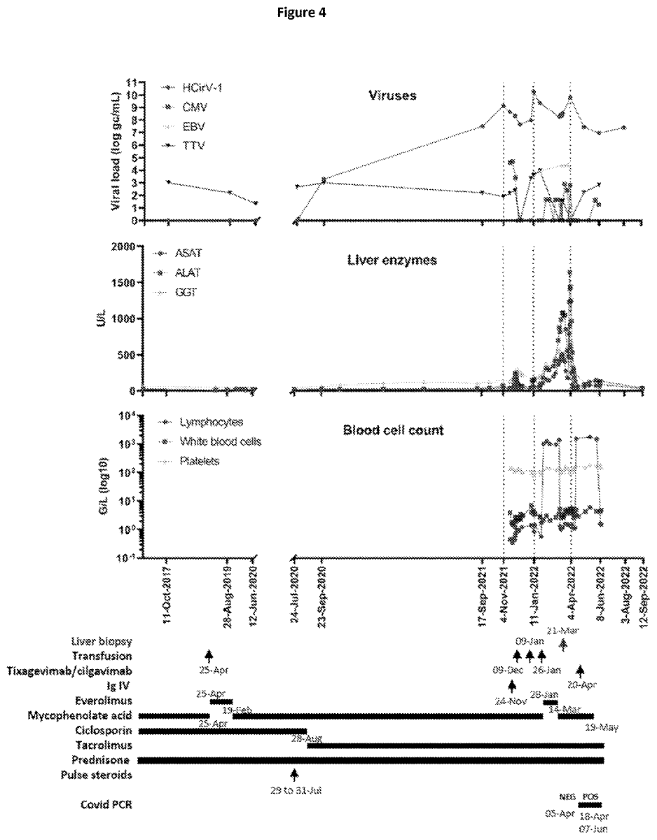

In November 2021, the patient was hospitalized for a CMV colitis with resistant virus requiring foscavir then maribavir treatment followed by letermovir long-term maintenance therapy. At this time cytolysis was detected (FIG. 4) without symptoms or liver abnormalities on ultrasound exam.

Four months after this hospitalization, her hepatitis worsened with transaminase serum levels increasing to up to 40 times the normal upper limit (FIG. 4) leading to readmission. The patient was asymptomatic except recent weight loss, without biological severity as the prothrombin time level was normal. Usual infections were ruled out, including hepatitis A, B, C, E, HIV, CMV, HSV, VZV, Human Herpes virus 6, adenovirus, enterovirus (in blood and stool), Parvovirus B19, toxoplasmosis, syphilis and leptospirosis. Only the EBV PCR was positive (104.4 genome copies/mL of blood). There was no evidence for a timeline relationship between the evolution of liver enzymes and the patient's various medications. She reported no use of paracetamol, alcohol, illicit drug or phytotherapy. The cardiac ultrasound did not show cardiac failure. There was no cardiac arrythmia, no vascular or biliary abnormalities at liver ultrasound and Doppler exam, CT scan and biliary MRI. Tests for auto-immune abnormalities were negative. Finally, ceruleoplasminemia was normal. A liver biopsy was then performed. Pathological examination showed lobular hepatitis, slightly inflammatory, without epithelioid granuloma, significant portal inflammation and fibrosis, or lymphoproliferation. Ziehl, EBV, CMV and adenovirus stainings were negative. Bacteriological examinations of the biopsy sample (including mycobacterial cultures and PCR, broad range 16S ribosomal RNA gene PCR) were negative. HSV-1, -2, CMV, Adenovirus and Enterovirus PCR were negative and EBV PCR was positive (135 gc/g DNA, cycle threshold=35) but was not ascribed to acute hepatitis. Overall, the lymphocytic pattern of inflammation in contrast of the lack of eosinophil infiltration, vascular or biliary injuries, and auto-immune liver disease (no plasmocytes) strongly suggested viral hepatitis. The extend of both centro-lobular necrosis and resorption by macrophages hinted for a recent and important liver cytolysis. Following these first-line negative results, mNGS was conducted on the liver biopsy and showed a novel circovirus (HCirV-1). Further exploration and quantification of HCirV-1 by qPCR was performed on liver tissue, stool, bronchoalveolar lavage, urine, and saliva (Table 1). Review and testing of the patient's historical blood samples was also done (Table 2).

TABLE 1
Liver biopsy, stool, urine, saliva and bronchoalveolar (BAL) samples
tested by qPCR for the detection and quantification of HCirV-1

| Sample | Sampling | qPCR Ct | Cp/mL (urine, saliva, BAL) |
|---|---|---|---|
| type | date | values | or Cp/gram (biopsy, stool) |
| Stool | 19 Nov. 2021 | 23.5 | 3.12E+05 |
| BAL | 6 Jan. 2022 | 24.8 | 3.24E+05 |
| Stool | 27 Jan. 2022 | 25.5 | 7.98E+04 |
| Liver biopsy | 21 Mar. 2022 | 16.4 | 3.57E+08 |
| Stool | 1 Apr. 2022 | 23.7 | 2.68E+05 |
| Stool | 11 May 2022 | 28.6 | 1.06E+04 |
| Urine | 8 Jun. 2022 | 26.4 | 1.13E+05 |
| Saliva | 8 Jun. 2022 | 26.0 | 1.45E+05 |
| Stool | 9 Jun. 2022 | 27.2 | 2.60E+04 |

TABLE 2
Blood, plasma and serum samples tested by qPCR
for the detection and quantification of HCirV-1

| Sample | Sampling | qPCR Ct | |
|---|---|---|---|
| type | date | values | Cp/mL |
| Serum | 11 Oct. 2017 | neg | neg |
| Serum | 28 Aug. 2019 | neg | neg |
| Serum | 12 Jun. 2020 | neg | neg |
| Serum | 24 Jul. 2020 | neg | neg |
| Serum | 23 Sep. 2020 | 32.4 | 2.00E+03 |
| Plasma | 17 Sep. 2021 | 17.9 | 3.24E+07 |
| Serum | 5 Nov. 2021 | 12.4 | 1.25E+09 |
| Serum | 18 Nov. 2021 | 13.9 | 4.38E+08 |
| Serum | 30 Nov. 2021 | 15.1 | 2.07E+08 |
| Serum | 10 Dec. 2021 | 17.4 | 4.49E+07 |
| Serum | 5 Jan. 2022 | 16.3 | 9.19E+07 |
| Serum | 12 Jan. 2022 | 8.4 | 1.72E+10 |
| Serum | 25 Jan. 2022 | 11.5 | 2.19E+09 |
| Plasma | 10 Mar. 2022 | 15.3 | 1.77E+08 |
| Serum | 14 Mar. 2022 | 14.6 | 2.77E+08 |
| Plasma | 16 Mar. 2022 | 15.1 | 2.08E+08 |
| Serum | 18 Mar. 2022 | 14.3 | 3.52E+08 |
| Blood | 4 Apr. 2022 | 10.0 | 5.81E+09 |
| Serum | 5 May 2022 | 18.1 | 2.73E+07 |
| Serum | 5 May 2022 | 17.2 | 4.92E+07 |
| Plasma | 8 Jun. 2022 | 19.2 | 1.36E+07 |
| Blood | 8 Jun. 2022 | 20.3 | 6.36E+06 |

As of June 2022, the patient's clinical condition is currently stable. We note an improvement of cytolysis. In this context of recent infectious diseases, immunosuppressive treatment has just been minimized with mycophenolate mofetil discontinuation (FIG. 4). We tried to ascertain possible sources of infection with HCirV-1. The patient reported contact with 2 cats, both of which had contact with birds and rodents. HCirV-1 PCR was negative in the faeces of her cats 2 months after the diagnosis. The patient denied any international travel. She did report a blood transfusion that occurred 17 months prior to the first HCirV-1 detection (FIG. 4).

The inventors searched for viral sequences in the liver of heart and lung double-graft recipient presenting with a profound liver cytolysis, and identified a new circovirus.

Damaged liver tissue was obtained from a biopsy and very deep sequencing (High Throughput Sequencing, or NGS) was performed.

Nucleic acids from the patient's liver biopsy were extracted using the geneLEAD VIII instrument (Diagenode), leading to a concentration of 15 ng/L. The RNA Integrity Number (RIN) was 2.9 after extraction. Two protocols were followed for Metagenomics Next-Generation Sequencing library preparation: (i) a cDNA library was constructed with the SMARTer Stranded Total RNA-Seq Kit-Pico Input Mammalian (Takara Bio, kit v.3). The procedure included a random reverse transcription of total RNA into first strand cDNA, a depletion of human ribosomal cDNA, and a final PCR amplification; (ii) a cDNA library was constructed using the SuperScript IV First-Strand Synthesis System kit (Invitrogen) according to the manufacturer's instructions with random primers, followed by MALBAC amplification (Yikon Genomics) and used as input for Illumina DNAPrep (Illumina, formerly Nextera DNA Flex) to generate the final NGS library.

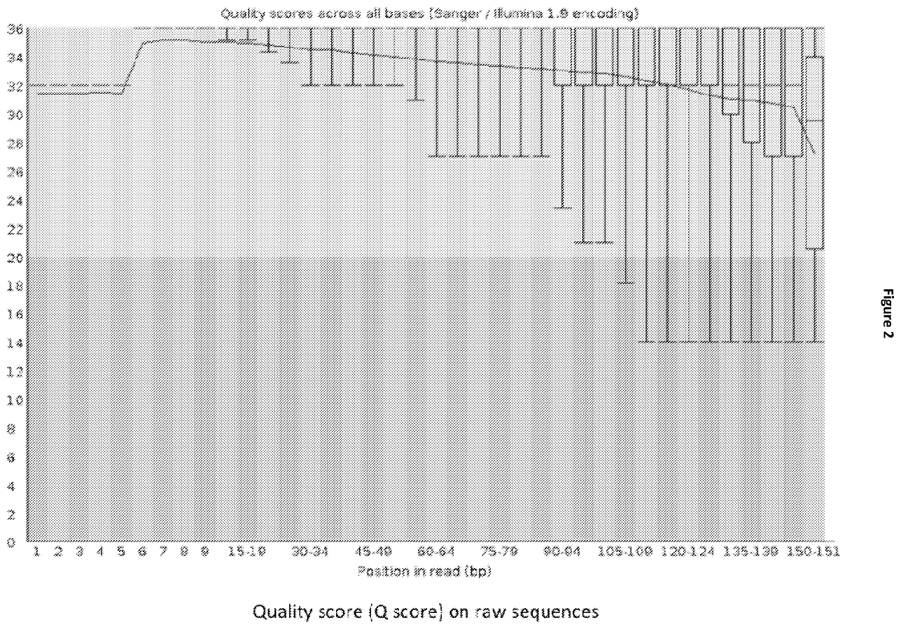

The RT/MALBAC/DNA Prep library was sequenced in 1×151 bp on a NextSeq500 instrument (Illumina) using the High Output flow cell, generating approximately 80 raw million reads. The vast majority of raw reads had quality scores (Q scores) above 20 (FIG. 2).

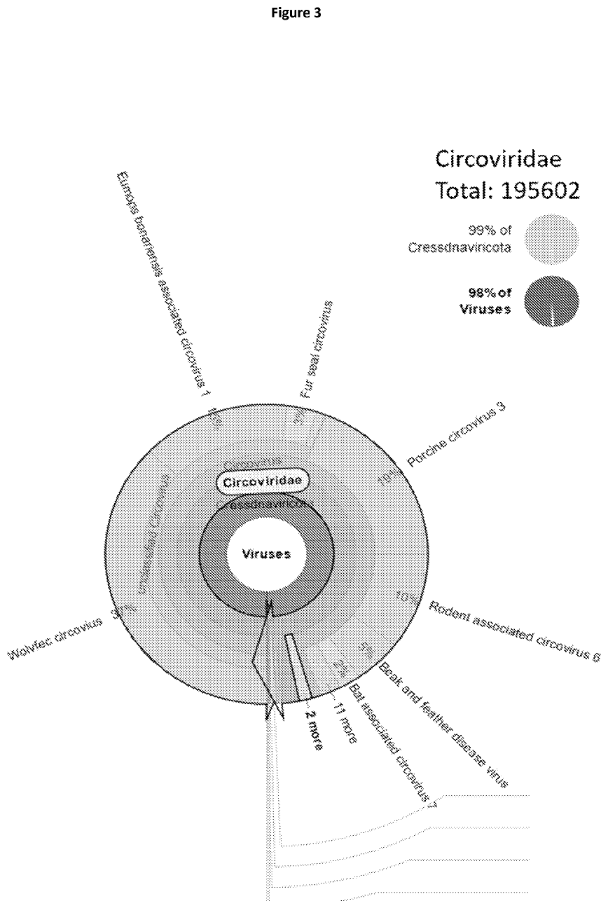

Raw reads from the enrichment-based sequencing were processed with an in-house bioinformatics pipeline (Microseek; Bigot et al., DOI: 10.3390/v14091990) comprising quality check and trimming, reads normalization, de novo assembly, and ORF prediction of contigs and singletons, followed by 3 levels of taxonomic assignation. The resulting viral hits with their associated taxonomic levels were depicted as a Krona (FIG. 3).

The longest capsid contig available (k141_422_flag=1_multi=91.0000_len=931_742_41_46_234) has a 39% identity at the amino acid level with the closest virus identified (Wolvfec circovirus Genbank QSX73454). Sequences identified as Circoviridae were then mapped onto appropriate reference sequences using GENEIOUS PRIME 2022.1.1 (Biomatters Ltd) to describe the full genome (FIG. 3). From the longest capsid contig used as seed, the full sequence of the capsid was reconstructed using an iterative mapping approach. The nucleotide sequence as well as the amino acid sequence of the reconstructed capsid are shown in SEQ ID NO: 1 and SEQ ID NO: 2.

A new species of circovirus was identified, named Human circovirus 1 (HCirV-1) within the genus Circovirus of the family Circoviridae. No pathogenic circovirus was previously known in humans.

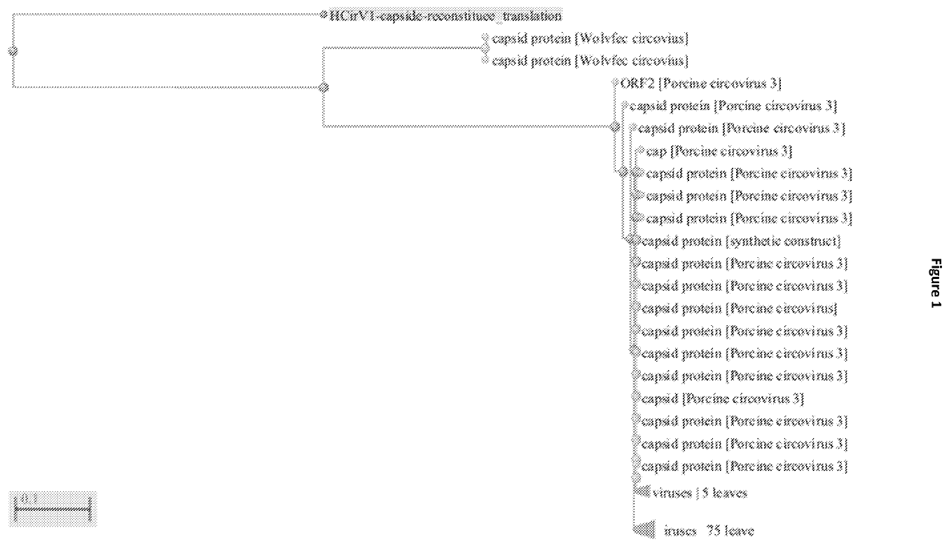

Based on the sequences, a phylogenetic tree was generated and is shown in FIG. 1. The phylogenetic analysis of the capsid gene (blast. ncbi. nlm. nih. gov/) shows that this virus corresponds to a new clade within the circovirus genus members.

The capsid of the novel human circovirus shows only 39% identity at the amino acid level with the closest circovirus, Wolvfec circovirus, Genbank QSX73454 shown below:

(SEQ ID NO: 3)
MRRYRRLRRR RRRRPRRPRY RRRIRRTWIR RPTAGTYYTK
KYSTMNIINL GKRESTKYWKVGHYTTSLKE WGSKWIWDYY
KILKMKITFY PQESNCFQHS LLWGHTVIDY
DGSWSTEGWLQDDPYANSST GRVWMSNKKH SRYFTPKPFL
VQTSSSYTGQ SLFYFTRNTP WLNCYDLDTKWGALLFSAYA
PADDTTPLYV QKSVWIRFKT VL.

On the contrary, there are numerous circoviruses known in animals (Beak and feather disease virus (BFDV), Porcine circovirus 1, 2, and 3 (PCV1-3)), including suspected hepatotropic viruses (Equine circovirus 1 strain). Hui et al., 2021 (https://doi.org/10.3390/v13050944). The nucleotide and amino acid sequences of the capsid protein of HCirV-1 are shown in SEQ ID NO: 1 and SEQ ID NO: 2.

The number of viral sequences in the liver biopsy was very high (>1000 contigs and singletons) and no other viral or bacterial sequences was found by NGS, supporting the role of HCirV-1 in the disease.

### Description of Controls

We retrospectively performed control HCirV-1 qPCRs in stool and blood samples of immunocompromised or immunocompetent patients with or without hepatitis (Table 3). We also reviewed the results of mNGS routinely performed on liver biopsies for pathogen diagnosis since 2019 in our lab (Table 4).

TABLE 3
Controls characteristics for assessment of HCirV-1 presence in 36
blood and 20 stool samples performed with specific HCirV-1 qPCR

| | | Immunocompetent | Immunocompromised | |
|---|---|---|---|---|
| | Hepatitis^{a} | w/o hepatitis | w/o hepatitis | Total |
| Controls | (n = 16) | (n = 12) | (n = 28) | (n = 56) |
| Age, mean (range), years | 17(3-53) | 30(0.2-67) | 44(1-84) | 33(0.2-84) |
| Adults >15 years old, n(%) | 5(31) | 7(58) | 22(78) | 35(63) |
| Sex, female, n(%) | 4(25) | 7(58) | 11(39) | 22(39) |
| Samples tested, n(%) | | | | |
| blood | 10(62) | 8(66) | 18(64) | 36(64) |
| stool | 6(38) | 4(33) | 10(36) | 20(36) |
| Immunological status, n(%) | | | | |
| immunocompetent | 5(31) | 12(100) | 0 | 17(30) |
| HIV seropositive | 0 | 0 | 2(7) | 2(4) |
| Solid organ transplant | 1(6) | 0 | 12(43) | 13(23) |
| Primary immune deficiency | 10(63) | 0 | 9(32) | 19(34) |
| HSCT or | 0 | 0 | 5(18) | 5(9) |
| immunosuppressive | | | | |
| therapy | | | | |

Table legend:
^{a}= Hepatomegaly and/or liver cytolysis;
w/o = without;
HSCT = Hematopoietic Stem Cell Transplant

TABLE 4
Controls characteristics for assessment of HCirV-1
presence in 57 liver biopsies samples screened
with metagenomic Next Generation Sequencing

| Controls | Total (n = 57) | |
|---|---|---|
| Age, mean (range), years | 13 | (0.3-68) |
| Adults >15 years old, n(%) | 10 | (18) |
| Sex, female, n(%) | 20 | (35) |
| Immunological status, n(%) | | |
| immunocompetent | 5 | (9) |
| HIV seropositive | 2 | (4) |
| Solid organ transplant | 5 | (9) |
| Primary immune deficiency | 25 | (44) |
| HSCT or immunosuppressive therapy | 20 | (35) |

### Nucleic Acids Extraction

Nucleic acids from the patient's liver biopsy were extracted using the geneLEAD VIII instrument (Diagenode). Nucleic acids from 200 L of plasma, serum, whole blood, bronchoalveolar lavage (BAL) and stool were extracted using the Emag instrument (Biomérieux).

mNGS

cDNA was generated from patient's liver biopsy using the SuperScript IV First-Strand Synthesis System kit (Invitrogen). MALBAC amplification (Yikon Genomics) was used as previously described [Regnault B, Bigot T, Ma L, Perot P, Temmam S, Eloit M. Deep Impact of Random Amplification and Library Construction Methods on Viral Metagenomics Results. Viruses 2021; 13:253] as input for Illumina DNAPrep (Illumina, formerly Nextera DNA Flex). The library was sequenced in 1×151 bp on a NextSeq500 instrument (Illumina) generating approximately 80 raw million reads. Raw reads were processed with an in-house bioinformatics pipeline using the RVDB-prot reference viral database. Bigot T, Temmam S, Pérot P, Eloit M. RVDB-prot, a reference viral protein database and its HMM profiles. F1000Res 2019; 8:530.

qPCR HCirV-1

The following PCR primers were designed in the capsid gene of HCirV-1: HCirV1-Fw1: 5-ACCTGGATGGACCCTGGAAT-3 (SEQ ID NO: 4); HCirV1-Rv1: 5-AGAGTTCCACCAGGTTCTGC-3 (SEQ ID NO: 5) (194 bp). Quantitative PCRs were done in SYBR Green format with 45 cycles of amplification with an annealing temperature of 58° C. Positive amplicons after 45 cycles were purified on gel, confirmed by Sanger sequencing and used for serial-dilutions to generate standard curves for the calculation of viral loads.

### Acquisition of a Complete Genome

The full genome of HCirV-1 was obtained by sequencing the patient's plasma of Jan. 12, 2022 (harbouring 1010 viral copies/mL), after a pre-amplification with the phi29 polymerase (WTA, Qiagen). An iterative mapping approach using Geneious Prime 2022.1.1 (Biomatters Ltd) was applied to perform the final assembly. The complete genome sequence was deposited to GenBank under accession number ON677309.

Identification of a novel circovirus in the liver biopsy NGS was used to analyze viral sequences from the liver biopsy specimen. A total of 1011 sequences of the Circoviridae family were identified, corresponding to the capsid and Rep genes. From a plasma sample taken at the peak of viremia, we derived a consensus full-genomic sequence (2021 pb) of the virus, which we refer to as HCirV-1 (Genbank ON677309) and confirmed that the sequence was identical to partial sequences of the capsid gene found in the liver. We conducted phylogenetic analyses on the two viral proteins, capsid and Rep of HCirV-1 and on representative animal circoviruses. The two analyses were congruent and showed that HCirV-1 defines a new clade at a basal position relative to the closest viral species (Wolvfec circovirus and Porcine circovirus 3).

### Phylogeny

Phylogenetic reconstructions were performed on the capsid protein sequence, on the replicase (Rep) protein sequence and on the complete nucleotide sequence of HCirV-1. Complete ORFs of the capsid and replicase genes were aligned along with other representative sequences of circoviruses using MAFFT (Multiple Alignment using Fast Fourier Transform) aligner under the LINS-i parameter. Maximum-likelihood phylogenetic reconstruction was performed with PhyML implemented through the NGPhylogeny portal. Lemoine F, Correia D, Lefort V, et al. NGPhylogeny.fr: new generation phylogenetic services for non-specialists. Nucleic Acids Research 2019; 47: W260-W265. Nodal support was evaluated using the “Approximate Bayes branch supports”.

TTV and EBV qPCR

TTV DNA load was performed using the TTV R-GENER kit (Biomérieux, Marcy-l'Etoile, France) as previously described. Kulifaj D, Durgueil-Lariviere B, Meynier F, et al. Development of a standardized real time PCR for Torque teno viruses (TTV) viral load detection and quantification: A new tool for immune monitoring. J Clin Virol 2018; 105:118-127. EBV DNA load was performed using EBV R-GENER kit (Biomérieux, Marcy l'Etoile, France). Real-time PCR amplification was performed on an AB7500 platform (Applied Biosystems, Waltham, MA, United States).

We designed and conducted a quantitative PCR (qPCR) to assess changes in the viral load in plasma and other biological samples taken from the case since 2017. We also evaluated the torque teno virus (TTV) load, a commensal virus whose level of viremia is well correlated with the immune status of graft recipients. Jaksch P, Görzer I, Puchhammer-Stöckl E, Bond G. Integrated Immunologic Monitoring in Solid Organ Transplantation: the Road Towards Torque Teno Virus-guided Immunosuppression. Transplantation 2022. TTV DNA load varied between 0 and 3.99 log cp/ml from 2017 to the present. This indicates a low level of immune suppression [Redondo N, Navarro D, Aguado J M, Fernandez-Ruiz M. Viruses, friends, and foes: The case of Torque Teno Virus and the net state of immunosuppression. Transpl Infect Dis 2022; 24: e13778] and TTV viremia peaks were not associated with peaks in cytolysis. In contrast, the first presence of HCirV-1 in blood could be dated in September 2020 (103.3 gc/mL), which is 2 months after steroids pulse, while the previous sample (July 2020) was negative. Nevertheless, it is noteworthy that this low level of HCirV-1 on September 2020 were questionable as the tubes were opened at the same time as other ones harboring up to 109.1 gc/mL, which could have resulted in cross-contamination. The next available sample was taken in September 2021 and was strongly positive (107.5 gc/mL). Thereafter, loads varied between 108.3 and 1010.2 gc/mL. HCirV-1 loads were high only during the period of pronounced cytolysis.

### Assessment of HCirV-1 Presence in Controls

No positive sample was detected in controls tested using the same specific qPCR primers for assessing HCirV-1 presence. A total of 36 blood and 20 stool samples from 56 patients were tested (Table 3). Among these patients 19 (34%), 17 (30%), 13 (23%), 5 (9%) and 2 (4%) were respectively primitively immunocompromised, immunocompetent, solid organ transplant recipients, hematopoietic stem cell transplant recipients or receiving immunosuppressive treatment and HIV positive. Among 16 patients with hepatic cytolysis (Table 3), 8 were of unknown etiology. Control biopsies routinely screened by mNGS since 2019 did not show HCirV-1 sequences among 57 patients with hepatitis and suspected infectious origin 25 (44%), 20 (35%), 5 (9%), 5 (9%) and 2 (4%) were respectively primitively immunocompromised, hematopoietic stem cell transplant recipients or receiving immunosuppressive treatment, immunocompetent, solid organ transplant recipients and HIV positive (Table 4). We show here a temporal association between high load of a novel human virus (HCirV-1) in liver and blood and hepatitis in a heart-lung transplant 61 years-old woman. The patient received a standard immunosuppressive regimen and TTV viremia was not in favor of a high level of immune suppression. These results should be evaluated with regards to Hill's criteria for causality. Hill A B. The Environment and Disease: Association or Causation? Proc R Soc Med 1965; 58:295-300. The specificity of the association was demonstrated by the absence of any other virus using agnostic mNGS in the liver biopsy except EBV detected by PCR, which was not considered to be responsible for such cytolysis given its low concentration in the liver biopsy, the concomitant higher replication in the blood (104.4 genome copies/mL) and the negative result of EBV staining on liver biopsy. HCirV-1 was not evidenced by qPCR neither in 40 controls without hepatitis, nor in 16 controls with hepatitis of known (50%) or unknown (50%) etiology (Table 3). In addition, HCirV-1 was not detected among 57 additional hepatitis cases of unknown etiology at the time of prescription investigated by mNGS (unpublished results, Table 4). Altogether, we did not detect the HCirV-1 genome in 113 controls.

Circoviruses are DNA viruses. Importantly, HCirV-1 transcripts were detected in the nucleus of 2% of liver cells by specific ISH. ISH results were therefore consistent with viral expression and probably virus replication. Given the lytic cycle of circoviruses, the results strongly support the causative role of HCirV-1 in hepatitis by targeting and lysing hepatocytes, and also as a likely trigger of immune cytotoxic responses.

Animal circoviruses like Porcine Circovirus (PCV) 1 to 4 could be responsible of reproductive failure, dermatitis, nephropathy and respiratory diseases [Sirisereewan C, Thanawongnuwech R, Kedkovid R. Current Understanding of the Pathogenesis of Porcine Circovirus 3. Pathogens 2022; 11:64; Wang D, Mai J, Yang Y, Xiao C-T, Wang N. Current knowledge on epidemiology and evolution of novel porcine circovirus 4. Veterinary Research 2022; 53:38] which were not found here. Association of circoviruses with hepatitis has been demonstrated in a horse [Hui A, Altan E, Slovis N, Fletcher C, Deng X, Delwart E. Circovirus in Blood of a Febrile Horse with Hepatitis. Viruses 2021; 13:944]. After inoculation of PCV3 in piglets, viremia progressively increases up to 108.9 copies/mL, which is reminiscent of our findings, but generally peaks around 21 days post infection (dpi) contrarily to the more delayed peak we have identified. Pathological lesions and PCV3-antigens are detected in the liver (including the presence of white-grey nodules and necrosis with PCV-3 antigen detected in liver lobular stroma, the hepatic sinus antral wall, and the cytoplasm in the sinus (Fujiwara K, Kojima H, Yasui S, et al. Hepatitis A viral load in relation to severity of the infection. Journal of Medical Virology 2011; 83:201-207) and in other organs (lung, heart, kidney, lymph nodes, spleen, and small intestine) [Jiang H, Wang D, Wang J, et al. Induction of Porcine Dermatitis and Nephropathy Syndrome in Piglets by Infection with Porcine Circovirus Type 3. Journal of Virology 2019; 93: e02045]. Interestingly, PCV3 viremia could still be observed at 140 dpi [Opriessnig T, Prickett J R, Madson D M, et al. Porcine circovirus type 2 (PCV2)-infection and reinoculation with homologous or heterologous strains: virological, serological, pathological and clinical effects in growing pigs. Vet Res 2010; 41:31], which is indicative of circoviruses ability to sustain long term infection as described in the patient. Regarding plausibility, there was no temporal link for a drug etiology or pathological evidence. Common hepatitis virus infections have been ruled out. EBV was positive, however the pathogenic role of EBV was ruled out because EBV viral load remained stable around 104 gc/ml (FIG. 4) and EBER staining was negative. The first detection of HCirV-1 was prior to the liver pathology, more precisely the first and second viral load peaks were respectively prior to the first and second cytolysis peaks, and the third viral load peak was concomitant to the cytolysis peak (FIG. 4). The high viral load of HCirV-1 (1010 gc/ml) is also in favor of the causative role of HCirV-1 in hepatitis. Indeed, a strong relationship between high virus load and disease severity has been descried for HBV. [Yu S J, Kim Y J. Hepatitis B viral load affects prognosis of hepatocellular carcinoma. World J Gastroenterol 2014; 20:12039-12044], HAV [Fujiwara K, Kojima H, Yasui S, et al. Hepatitis A viral load in relation to severity of the infection. Journal of Medical Virology 2011; 83:201-207], or adenovirus in hematopoietic stem cell transplant recipients [Lion T, Baumgartinger R, Watzinger F, et al. Molecular monitoring of adenovirus in peripheral blood after allogeneic bone marrow transplantation permits early diagnosis of disseminated disease. Blood 2003; 102:1114-1120]. Hence, viral load influences organ pathology [Funk G A, Gosert R, Hirsch H H. Viral dynamics in transplant patients: implications for disease. The Lancet Infectious Diseases 2007; 7:460-472]. As of today, we have not identified the source of infection. Specifically, we do not know if it was due to the spillover of an animal virus or if this is the first identified case of an uncovered human virus. HCirV-1 has never been identified previously as part of the human virome. We anticipate that it could be of animal origin, possibly from a dietary source in a similar manner as Hepatitis E virus. Phylogenetic analysis places this virus at distance of closest known viruses, which are themselves hosted by a wild exotic animal that cannot be a source for the patient. Development of a specific antibody test will be instrumental to decipher the ecology of the virus and better understand mechanisms of transmission.

As shown in Table 2, HCirV-1 was shed in saliva, urine and stools and therefore can possibly be transmitted to contacts, which remains to be investigated.

### In Situ Hybridization

ViewRNA ISH Tissue Assay Kit 2-plex (Thermo Fisher Scientific) was used to detect RNA sequences of HCirV-1 as a hallmark of virus replication. Cocktails of 12 and 20 custom bDNA probes were designed to target the capsid and Rep genes of HCirV-1, respectively, and were revealed by a red signal (as the “probe type 1” of the kit). A mix of control probes targeting human GAPDH, ACTB and PPI transcripts was revealed by a blue signal (as the “probe type 6” of the kit). To unmask RNA targets, the tissue sections (after deparaffinization using xylene) were pretreated by 20 min heat pretreatment and 15 min protease digestion. Harris haematoxylin was used for counterstaining. The negative liver biopsy controls used for ISH included a no probe negative control from the patient infected with HCiV-1 as recommended by the manufacturer and a negative control from a different patient tested negative for HCirV-1 with mNGS (this slide underwent the entire procedure assay).

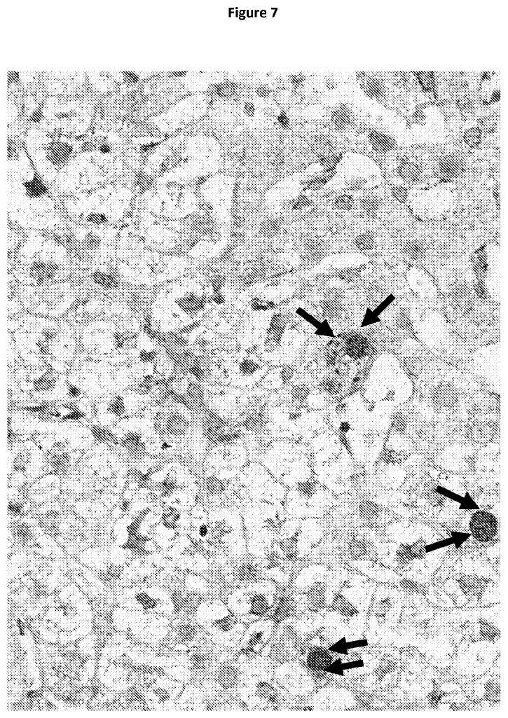

In situ hybridization (ISH) showed 2% of infected (red signal) hepatocytes in the liver biopsy (FIG. 7). Red labeling was strong in the nuclei and mild in the cytoplasm. Some hepatocytes were poorly red labeled in the nuclei and the red labeling was negative in the cytoplasm. Endothelial cells and lymphocytes were not infected. No red signal was recorded in the negative controls. The detection of HCirV-1 expression in liver cells by ISH, the exclusion of other hepatitis etiologies, and the temporal association of severe cytolysis with very high HCirV-1 loads in the liver and blood strongly suggest a causal link. Furthermore, our experience with the use of mNGS in immunocompromised patients (including transplant recipients), as well as the specific qPCR we performed on a representative cohort of these patients, proves that unlike TTV, HCirV-1 is not part of the commensal viral flora.

1. A recombinant nucleic acid comprising a human circovirus 1 (HCirV-1) nucleotide sequence, said sequence comprising SEQ ID NO: 10 or a fragment thereof of at least 10 successive nucleotides.

2. The recombinant nucleic acid of claim 1, wherein the nucleotide sequence is an HCirV-1 capsid or replicase sequence.

3. The recombinant nucleic acid of claim 2, wherein the nucleotide sequence comprises the capsid nucleotide sequence of SEQ ID NO: 1 or the replicase nucleotide sequence of SEQ ID NO: 11, or a fragment thereof of at least 10 successive nucleotides.

4. The recombinant nucleic acid of claim 1, wherein the nucleotide sequence encodes a protein having at least 70%, 75%, 80%, identity with SEQ ID NO: 2, with SEQ ID NO: 12, with SEQ ID NO: 16 or with SEQ ID NO: 17.

5. A kit for detecting an HCirV-1 nucleic acid comprising the recombinant nucleic acid of claim 3 and amplification and/or hybridization reagents.

6. A primer or probe comprising at least 10 successive nucleotides of SEQ ID NO: 1, SEQ ID NO: 10, or SEQ ID NO: 11 labeled with a fluorescent, radioactive, or enzymatic label.

7. The primer or probe of claim 6, which has a sequence selected from SEQ ID NO:4-9 or from SEQ ID NO: 13-15.

8. A kit comprising the primer or probe of claim 6 and amplification and/or hybridization reagents.

9. (canceled)

10. An in vitro method of detecting an HCirV-1 virus, said method comprising contacting the primer or probe of claim 6 with a biological sample and detecting the presence or absence of HCirV-1 nucleic acid in the sample.

11. (canceled)

12. (canceled)

13. An isolated protein or peptide comprising at least 15 amino acids of SEQ ID NO: 2 or of SEQ ID NO:12 or of SEQ ID NO: 16 or of SEQ ID NO: 17 or of variants thereof.

14. (canceled)

15. (canceled)

16. An isolated antibody that binds specifically to a HCirV-1 protein or peptide of claim 13.

17. The isolated antibody according to claim 16, wherein said protein has the amino acid sequence of SEQ ID NO: 2, SEQ ID NO: 12, SEQ ID NO: 16 or SEQ ID NO: 17.

18. (canceled)

19. (canceled)

20. An in vitro method of detecting an HCirV-1 comprising contacting the protein or peptide of claim 13 with a biological sample and detecting the presence or absence of HCirV-1 antibodies in the sample.

21. An immunogenic composition or vaccine composition comprising the protein or peptide of claim 13 and an adjuvant and/or a pharmaceutically acceptable vehicle.

22. An in vitro method of detecting an HCirV-1 comprising contacting the isolated antibody of claim 16 with a biological sample and detecting the presence or absence of HCirV-1 protein in the sample.

23. The recombinant nucleic acid of claim 1, wherein the nucleotide sequence comprises a fragment of SEQ ID NO: 10 of at least 15 successive nucleotides.

24. The recombinant nucleic acid of claim 1, wherein the nucleotide sequence comprises a fragment of SEQ ID NO: 10 of at least 20 successive nucleotides.

25. The primer or probe of claim 6, comprising at least 15 successive nucleotides of SEQ ID NO: 1, SEQ ID NO:10, or SEQ ID NO:11.

26. The primer or probe of claim 6, comprising at least 20 successive nucleotides of SEQ ID NO: 1, SEQ ID NO: 10, or SEQ ID NO: 11.


SEQUENCE LISTING (17 sequences)

SEQ ID NO: 1 - DNA, 645 bp, unidentified
  source 1..645: mol_type = genomic DNA
atggctagatataccctcaggaggagaagaccccgccccaggaggcgtgcccgcagatttcgacgaatcaggagacgccgcatttacataagacgtcccaccaataatggaacagttgtccaaaaattttgcaagactgaacccttcaccctccaaaaccaagattcaaccaatgccaaaaaatggatgcaaggctcttacacaaccaccctcagtgaatgggtttcctcaccaggctgggacttttataaaatcctaaaactcaaactaaccttccttcccttcaatgccacacagcgaatgaactactggcctataggaaacaccatcatagacctggatggaccctggaatgaacctaacacagtacaatcaaacctactctgcaacactgccacttccagactttggaaggctaacagaaagcatagcagatattttactcctcgccctctcatccatcttactggcaatactacttcttctcaagcatcagctctcctcccacgcagaacctggtggaactctgcagaatctactgtcaaatggggaggactcaagtactctcattacgttgaatataatcaaccagtcagatactggctcatcaaatcagcctggatcaaatggaaataccttctctaa

SEQ ID NO: 2 - protein, 214 aa, unidentified
MARYTLRRRRPRPRRRARRFRRIRRRRIYIRRPTNNGTVVQKFCKTEPFTLQNQDSTNAKKWMQGSYTTTLSEWVSSPGWDFYKILKLKLTFLPFNATQRMNYWPIGNTIIDLDGPWNEPNTVQSNLLCNTATSRLWKANRKHSRYFTPRPLIHLTGNTTSSQASALLPRRTWWNSAESTVKWGGLKYSHYVEYNQPVRYWLIKSAWIKWKYLL

SEQ ID NO: 3 - protein, 212 aa, unidentified
MRRYRRLRRRRRRRPRRPRYRRRIRRTWIRRPTAGTYYTKKYSTMNIINLGKRESTKYWKVGHYTTSLKEWGSKWIWDYYKILKMKITFYPQESNCFQHSLLWGHTVIDYDGSWSTEGWLQDDPYANSSTGRVWMSNKKHSRYFTPKPFLVQTSSSYTGQSLFYFTRNTPWLNCYDLDTKWGALLFSAYAPADDTTPLYVQKSVWIRFKTVL

SEQ ID NO: 4 - DNA, 20 bp, unidentified
  source 1..20: mol_type = genomic DNA
acctggatggaccctggaat

SEQ ID NO: 5 - DNA, 20 bp, unidentified
  source 1..20: mol_type = genomic DNA
agagttccaccaggttctgc

SEQ ID NO: 6 - DNA, 20 bp, unidentified
  source 1..20: mol_type = genomic DNA
ttcgacgaatcaggagacgc

SEQ ID NO: 7 - DNA, 20 bp, unidentified
  source 1..20: mol_type = genomic DNA
ggcagtgttgcagagtaggt

SEQ ID NO: 8 - DNA, 20 bp, unidentified
  source 1..20: mol_type = genomic DNA
caggagacgccgcatttaca

SEQ ID NO: 9 - DNA, 20 bp, unidentified
  source 1..20: mol_type = genomic DNA
cccattcactgagggtggtt

SEQ ID NO: 10 - DNA, 2021 bp, unidentified
  source 1..2021: mol_type = genomic DNA
cagtattacccggcacctcggaacctggaaccaggagcaagatagaggttttcaggtgtggcacttggcagcctatcaagatggatggacccgccaaaaagagcaaggtgctgtatggtgttcctgtggcagtgccgggtctgaatgtgaaatatgtttttgctgataatgtattgtactggcattgtgatgactgttataagttttggctggatgagaaggagattcaatcttatgtgtatttggatgcagcgggagagcccagtggcgaggtggtgttttacgataaacaactggacccaagaagagttggattgcttgctgcagtgtggcaaagagaaagcgaagtatctttgtatcggcagagaggttggaagcggcggtacccggcacttgcaaggatacgtgaatttcaagaacaagcgaagaatgagacaggtaaaggccttgcctggttttatgcgtgctcacttagaagcggccaaggggaccgagaagcaggcgagcgactactgcaagaaagacggcgattacctggagattggagagagtggctgtgcggggcgccgactggatttggagacagcagcgaagatactgactgagtctggcgggaacttgaagtgtgttgccgaggcttgtcccggggtttatataaagtatgggcggggtttgagggattacgcaagtgttatgaaattaaagcaacctcgcaattttaagagtactgtgattgttatatgtggggatcctggttgtgggaaaactagatatgttatggaggattgtagggagaaagggttaagcatgtattggaagccaaggggaatttggtgggatggctatgatggggaggatgtggtggtgtatgatgatttttatgggtgggttccttatgatgagatgttgagggttatggataggtatcctttgaaggttcctgttaaggggggttatgtggattttacatctaagattatatatgtgacaagtaatgtgaagcctgaggattggtatagtagtgagaatattaaggggaagttggagagtttgtttaggcggatcaatgtttatctggagggttttggcggggagatccgtgtgggaacgcctctgtatcctattaactattaataaagacaaacaatggttgaagttcaacggtttgtttatttggcgggtacaattggcgggaaggagggaaaaatggcgggagttactgactgggccgcacagccgccgcgggggtccccgacagggtcggggcgcgggggatcttttccggccgccccgttagggcaggaaaagcgtttacctggtcacttggggcgggtcacccccctcggagtcagtttatataagtttgcttagagaaggtatttccatttgatccaggctgatttgatgagccagtatctgactggttgattatattcaacgtaatgagagtacttgagtcctccccatttgacagtagattctgcagagttccaccaggttctgcgtgggaggagagctgatgcttgagaagaagtagtattgccagtaagatggatgagagggcgaggagtaaaatatctgctatgctttctgttagccttccaaagtctggaagtggcagtgttgcagagtaggtttgattgtactgtgttaggttcattccagggtccatccaggtctatgatggtgtttcctataggccagtagttcattcgctgtgtggcattgaagggaaggaaggttagtttgagttttaggattttataaaagtcccagcctggtgaggaaacccattcactgagggtggttgtgtaagagccttgcatccattttttggcattggttgaatcttggttttggagggtgaagggttcagtcttgcaaaatttttggacaactgttccattattggtgggacgtcttatgtaaatgcggcgtctcctgattcgtcgaaatctgcgggcacgcctcctggggcggggtcttctcctcctgagggtatatctagccatggtgccggt

SEQ ID NO: 11 - DNA, 888 bp, unidentified
  source 1..888: mol_type = genomic DNA
atgcagcgggagagcccagtggcgaggtggtgttttacgataaacaactggacccaagaagagttggattgcttgctgcagtgtggcaaagagaaagcgaagtatctttgtatcggcagagaggttggaagcggcggtacccggcacttgcaaggatacgtgaatttcaagaacaagcgaagaatgagacaggtaaaggccttgcctggttttatgcgtgctcacttagaagcggccaaggggaccgagaagcaggcgagcgactactgcaagaaagacggcgattacctggagattggagagagtggctgtgcggggcgccgactggatttggagacagcagcgaagatactgactgagtctggcgggaacttgaagtgtgttgccgaggcttgtcccggggtttatataaagtatgggcggggtttgagggattacgcaagtgttatgaaattaaagcaacctcgcaattttaagagtactgtgattgttatatgtggggatcctggttgtgggaaaactagatatgttatggaggattgtagggagaaagggttaagcatgtattggaagccaaggggaatttggtgggatggctatgatggggaggatgtggtggtgtatgatgatttttatgggtgggttccttatgatgagatgttgagggttatggataggtatcctttgaaggttcctgttaaggggggttatgtggattttacatctaagattatatatgtgacaagtaatgtgaagcctgaggattggtatagtagtgagaatattaaggggaagttggagagtttgtttaggcggatcaatgtttatctggagggttttggcggggagatccgtgtgggaacgcctctgtatcctattaactattaa

SEQ ID NO: 12 - protein, 295 aa, unidentified
MQRESPVARWCFTINNWTQEELDCLLQCGKEKAKYLCIGREVGSGGTRHLQGYVNFKNKRRMRQVKALPGFMRAHLEAAKGTEKQASDYCKKDGDYLEIGESGCAGRRLDLETAAKILTESGGNLKCVAEACPGVYIKYGRGLRDYASVMKLKQPRNFKSTVIVICGDPGCGKTRYVMEDCREKGLSMYWKPRGIWWDGYDGEDVVVYDDFYGWVPYDEMLRVMDRYPLKVPVKGGYVDFTSKIIYVTSNVKPEDWYSSENIKGKLESLFRRINVYLEGFGGEIRVGTPLYPINY

SEQ ID NO: 13 - DNA, 19 bp, unidentified
  source 1..19: mol_type = genomic DNA
ttagaagcggccaagggga

SEQ ID NO: 14 - DNA, 20 bp, unidentified
  source 1..20: mol_type = genomic DNA
cgctgctgtctccaaatcca

SEQ ID NO: 15 - DNA, 21 bp, unidentified
  source 1..21: mol_type = genomic DNA
ggagattggagagagtggctg

SEQ ID NO: 16 - protein, 180 aa, unidentified
NNGTVVQKFCKTEPFTLQNQDSTNAKKWMQGSYTTTLSEWVSSPGWDFYKILKLKLTFLPFNATQRMNYWPIGNTIIDLDGPWNEPNTVQSNLLCNTATSRLWKANRKHSRYFTPRPLIHLTGNTTSSQASALLPRRTWWNSAESTVKWGGLKYSHYVEYNQPVRYWLIKSAWIKWKYLL

SEQ ID NO: 17 - protein, 180 aa, unidentified
NNASIVQKFCKWEPISFQNTTNQNKWYIGSYTTNLKEWVTQPNWDLYKILKMKISFLPFTPTQKMPYWPIGNTIIDLDGPWPQPYTANSNLFCNTATSRSWYGNKKHSRYFTPRAVVNLTTTATTGEGTALLPRSTWWNCNNDSIIWGGLRFAHYIENGSNMAIRYHLQKSVWIKWKYLL